(19) United States
(12) Patent Application Publication
(10) Pub. No.: US 2025/0332248 A1
(43) Pub. Date: Oct. 30, 2025

(54) COMPOSITIONS AND METHODS FOR INCREASING VIRAL NUCLEOCAPSID PROTEIN DIMERIZATION

(71) Applicants: University of Vermont and State Agricultural College, Burlington, VT (US); The Board of Regents of the University of Texas System, Austin, TX (US)

(72) Inventors: Emily Bruce, Burlington, VT (US); Hannah Kubinski, Burlington, VT (US); Hannah Despres, Burlington, VT (US); Vineet D. Menachery, Galveston, TX (US); Bryan A. Johnson, Galveston, TX (US)

(73) Assignees: University of Vermont and State Agricultural College, Burlington, VT (US); The Board of Regents of The University of Texas System, Austin, TX (US)

(21) Appl. No.: 19/074,686

(22) Filed: Mar. 10, 2025

Related U.S. Application Data
(60) Provisional application No. 63/562,754, filed on Mar. 8, 2024.

(51) Int. Cl. A61K 39/215; A61K 39/00; A61P 37/04; C07K 14/005

(52) U.S. Cl. CPC A61K 39/215; A61P 37/04; C07K 14/005; A61K 2039/5254; C07K 2319/00; C12N 2770/20022; C12N 2770/20034; C12N 2770/20071

(57) ABSTRACT

The invention, in some aspects, relates to compositions comprising modified viral nucleocapsid (N) proteins and their encoding polynucleotides and methods of using such compositions and preparations to increase viral N protein dimerization and/or increase antigenicity of viral immunization preparations.

### RELATED APPLICATIONS

This application claims benefit under 35 U.S.C. § 119(e) of U.S. Provisional application Ser. No. 63/562,754 filed Mar. 8, 2024, the disclosure of which is incorporated by reference herein in its entirety.

### GOVERNMENT INTEREST

This invention was made with government support under grants P30GM118228-04 and 1R01AI153602-01 awarded by the National Institutes of Health. The government has certain rights in the invention.

### FIELD OF THE INVENTION

The invention relates, in part, to compositions and methods for increasing virus nucleocapsid (N) protein dimerization.

### Reference to an Electronic Sequence Listing

The contents of the electronic sequence listing (Sequence_Listing.xml; Size: (73,300 bytes; Date of Creation: Mar. 6, 2025) is herein incorporated by reference in its entirety.

### BACKGROUND

The coronavirus disease of 2019 (COVID-19) pandemic originated from the emergence of Severe Acute Respiratory Syndrome Coronavirus 2 (SARS-COV-2) in late 2019 [Cucinotta, D. & Vanelli, M. Acta Biomed. Atenei Parm. 91, 157-160 (2020)]. Subsequent worldwide spread and sustained transmission over the past four years, combined with near real-time access to viral genomic surveillance data, has revealed a detailed picture of SARS-COV-2 evolution in the human population. Individual mutations that conferred an evolutionary advantage were quickly selected and maintained in viral lineages, leading to the emergence of novel Variants of Concern distinguished by increased immune evasion, disease burden or infectivity. Understanding the biological function of the individual mutations that led to the emergence of novel variants of concern has important implications for public health consequences as well as our fundamental understanding of basic coronavirus biology.

Most studies concerning these mutations of concern have focused on genetic changes within the spike(S) protein, due to concerns that sufficiently novel spike proteins can allow viral escape from the immune memory induced by vaccination or prior infection [Plante, J. A. et al. Nature 592, 116-121 (2021); Johnson, B. A. et al. Nature 591, 293-299 (2021); Liu, Y. et al. Cell Rep. 39, (2022); and Vu, M. N. et al. Proc. Natl. Acad. Sci. 119, e2205690119 (2022)]. However, mutations elsewhere in the viral genome can play key roles in viral replication and pathogenesis [McGrath, M. E. et al., Proc. Natl. Acad. Sci. 119, e2204717119 (2022); Carabelli, A. M. et al. Nat. Rev. Microbiol. 21, 162-177 (2023); and Johnson, B. A. et al. PLOS Pathog. 18, e1010627 (2022)].

### Summary of Aspects of the Invention

According to an aspect of the invention, a method of increasing antigenicity of a viral immunization preparation is provided, the method comprising: including in the viral immunization preparation a modified viral nucleocapsid (N) protein or a sequence encoding the modified viral N protein, wherein the modified viral N protein includes an amino acid substitution (also referred to herein as a “point mutation”) in a linker region of a viral N protein. In some embodiments, when the modified viral N protein is expressed, the presence of the mutation increases a level of N-N dimers in the viral immunization preparation compared to a control level of N-N dimers and increases the level of antigenicity of the viral immunization preparation compared to a control level of antigenicity. In some embodiments, the control antigenicity is a level of antigenicity in an essentially similar viral immunization preparation that does not include the modified viral N protein. In some embodiments, the control level of N-N dimers is a level of N-N dimers in a substantially similar viral immunization preparation in the absence of the modified viral N protein. In some embodiments, the increased level of the N-N dimers increases the amount of N antigen in the viral immunization preparation. In some embodiments, the viral N protein is a coronavirus N protein. In some embodiments, the viral N protein is a Beta coronavirus protein. In some embodiments, the viral N protein is a Severe Acute Respiratory Syndrome coronavirus 2 (SARS-COV-2) protein, a Severe Acute Respiratory Syndrome coronavirus 1 (SARS-COV-1) protein, or a Middle Eastern Respiratory syndrome coronavirus (MERs CoV) protein. In some embodiments, the viral N protein is a SARS-COV protein. In some embodiments, the viral N protein is a SARs-COV-2 Delta protein, SARs-COV-2 Beta protein, or a SARs-COV-2 Iota protein. In some embodiments, the viral immunization preparation further includes at least one additional independently selected viral protein. In some embodiments, the at least one additional independently selected viral protein is one of more of a viral spike protein, a viral capsid protein, a viral envelope protein, and a viral membrane protein. In some embodiments, the viral immunization preparation includes a live attenuated viral immunization preparation or a whole-inactivated viral immunization preparation. In some embodiments, the viral immunization preparation includes a sequence encoding the viral N protein. In some embodiments, the amino acid sequence of the viral N protein is SEQ ID NO: 1 or a functional variant of SEQ ID NO: 1 with one or more amino acid substitutions (also referred to herein as one of more point mutations) including a glycine→cysteine (G→C) substitution or an arginine→cystine (R→C) substitution in the viral N protein. In some embodiments, the substitution is at or corresponds to amino acid G215 of SEQ ID NO: 1, or at a glycine in the functional variant of SEQ ID NO: 1 at a position that corresponds to the G215 in SEQ ID NO: 1. In some embodiments, the amino acid sequence of the modified N protein is SEQ ID NO: 2, or a functional variant of SEQ ID NO: 2. In some embodiments, the substitution is at or corresponds to amino acid G243 of SEQ ID NO: 1, or at a glycine in the functional variant of SEQ ID NO: 1 at a position that corresponds to the G243 in SEQ ID NO: 1. In some embodiments, the amino acid sequence of the modified N protein is SEQ ID NO: 3, or a functional variant of SEQ ID NO: 3. In some embodiments, the substitution is at amino acid R185 of SEQ ID NO: 1, or at an arginine in the functional variant of SEQ ID NO: 1 at a position that corresponds to the R185 in SEQ ID NO: 1. In some embodiments, the amino acid sequence of the modified N protein is SEQ ID NO: 4, or a functional variant of SEQ ID NO: 4. In some embodiments, the modified N protein has the amino acid sequence of SEQ ID NO: 5 or a functional variant of SEQ ID NO: 5. In some embodiments, the modified N protein has the amino acid sequence of SEQ ID NO: 6 or a functional variant of SEQ ID NO: 6. In some embodiments, the modified N protein has the amino acid sequence of SEQ ID NO: 7 or a functional variant of SEQ ID NO: 7. In some embodiments, the modified N protein has the amino acid sequence of SEQ ID NO: 8 or a functional variant of SEQ ID NO: 8.

According to another aspect of the invention, a method of increasing a level of N-N dimers in a virus particle is provided, the method comprising including in the virus particle, a modified viral nucleocapsid (N) protein including an amino acid substitution (also referred to herein as a point mutation) in a linker region of a viral N protein, wherein the presence of the substitution increases a level of N-N dimers in the viral particle compared to a control level of N-N dimers. In some embodiments, the control level of N-N dimers is a level of N-N dimers in a substantially similar viral particle in the absence of the modified viral N protein. In some embodiments, the viral N protein is a coronavirus N protein. In some embodiments, the viral N protein is a Beta coronavirus protein. In some embodiments, the viral N protein is a Severe Acute Respiratory Syndrome coronavirus 2 (SARS-COV-2) protein, a Severe Acute Respiratory Syndrome coronavirus 1 (SARS-COV-1) protein, or a Middle Eastern Respiratory syndrome coronavirus (MERs CoV) protein. In some embodiments, the viral N protein is a SARS-COV protein. In some embodiments, the increase in the level of N-N dimers in the virus particle increases the antigenicity of the virus particle compared to a control virus particle. In some embodiments, the control virus particle is a viral particle not including the increased level of N-N dimers. In some embodiments, the amino acid sequence of the viral N protein is SEQ ID NO: 1 or a functional variant of SEQ ID NO: 1. In some embodiments, the substitution is at or corresponds to amino acid G215 of SEQ ID NO: 1, or at a glycine in the functional variant of SEQ ID NO: 1 at a position that corresponds to the G215 in SEQ ID NO: 1. In some embodiments, the amino acid sequence of the modified N protein is SEQ ID NO: 2, or a functional variant of SEQ ID NO: 2. In some embodiments, the amino acid substitution (also referred to herein as the point mutation) is at or corresponds to amino acid G243 of SEQ ID NO: 1, or at a glycine in the functional variant of SEQ ID NO: 1 at a position that corresponds to the G243 in SEQ ID NO: 1. In some embodiments, the amino acid sequence of the modified N protein is SEQ ID NO: 3, or a functional variant of SEQ ID NO: 3. In some embodiments, the substitution (also referred to herein as a point mutation) is at amino acid R185 of SEQ ID NO: 1, or at an arginine in the functional variant of SEQ ID NO: 1 at a position that corresponds to the R185 in SEQ ID NO: 1. In some embodiments, the amino acid sequence of the modified N protein is SEQ ID NO: 4, or a functional variant of SEQ ID NO: 4. In some embodiments, the modified N protein has the amino acid sequence of SEQ ID NO: 5 or a functional variant of SEQ ID NO: 5. In some embodiments, the modified N protein has the amino acid sequence of SEQ ID NO: 6 or a functional variant of SEQ ID NO: 6. In some embodiments, the modified N protein has the amino acid sequence of SEQ ID NO: 7 or a functional variant of SEQ ID NO: 7. In some embodiments, the modified N protein has the amino acid sequence of SEQ ID NO: 8 or a functional variant of SEQ ID NO: 8.

According to another aspect of the invention, a composition including a modified viral nucleocapsid (N) protein or a functional fragment thereof is provided, wherein the modified viral N protein and the functional fragment thereof include an amino acid substitution (also referred to herein as a point mutation) in a linker region of the viral N protein. In some embodiments, the viral N protein is a coronavirus N protein. In some embodiments, the viral N protein is a Betacoronavirus N protein. In some embodiments, the viral N protein is a Severe Acute Respiratory Syndrome coronavirus 2 (SARS-COV-2) N protein, a Severe Acute Respiratory Syndrome coronavirus 1 (SARS-COV-1) N protein, or a Middle Eastern Respiratory syndrome coronavirus (MERs CoV) N protein. In some embodiments, the viral N protein is a SARS-COV protein. In some embodiments, the amino acid sequence of the viral N protein is SEQ ID NO: 1, or a functional variant of SEQ ID NO: 1. In some embodiments, the amino acid substitution (also referred to herein as the point mutation) is a glycine→cysteine (G→C) substitution or an arginine→cystine (R→C) substitution in the viral N protein. In some embodiments, the amino acid substitution (also referred to herein as the point mutation) is at amino acid G215 of SEQ ID NO: 1, or at a glycine in the functional variant of SEQ ID NO: 1 at a position that corresponds to the G215 in SEQ ID NO: 1. In some embodiments, the amino acid sequence of the modified N protein is SEQ ID NO: 2, or a functional variant of SEQ ID NO: 2. In some embodiments, the amino acid substitution (also referred to herein as the point mutation) is at amino acid G243 of SEQ ID NO: 1, or at a glycine in the functional variant of SEQ ID NO: 1 at a position that corresponds to the G243 in SEQ ID NO: 1. In some embodiments, the amino acid sequence of the modified N protein is SEQ ID NO: 3, or a functional variant of SEQ ID NO: 3. In some embodiments, the amino acid substitution (also referred to herein as the point mutation) is at amino acid R185 of SEQ ID NO: 1, or at an arginine in the functional variant of SEQ ID NO: 1 at a position that corresponds to the R185 in SEQ ID NO: 1. In some embodiments, the amino acid sequence of the modified N protein is SEQ ID NO: 4, or a functional variant of SEQ ID NO: 4. In some embodiments, the modified N protein has the amino acid sequence of SEQ ID NO: 5 or a functional variant of SEQ ID NO: 5. In some embodiments, the modified N protein has the amino acid sequence of SEQ ID NO: 6 or a functional variant of SEQ ID NO: 6. In some embodiments, the modified N protein has the amino acid sequence of SEQ ID NO: 7 or a functional variant of SEQ ID NO: 7. In some embodiments, the modified N protein has the amino acid sequence of SEQ ID NO: 8 or a functional variant of SEQ ID NO: 8.

According to another aspect of the invention, a fusion protein is provided, wherein the fusion protein includes a modified N protein composition of any embodiment of any aforementioned aspects of the invention.

According to another aspect of the invention, a cell that includes a fusion protein of an aforementioned aspect of the invention is provided.

According to another aspect of the invention, a polynucleotide sequence encoding a fusion protein of any aforementioned aspect of the invention is provided.

According to another aspect of the invention, a cell that includes a polynucleotide sequence any aforementioned aspect of the invention is provided.

According to another aspect of the invention, a viral immunization preparation is provided, wherein the preparation includes a modified N protein composition, or a functional fragment thereof, of any aforementioned aspect of the invention.

According to another aspect of the invention, a viral particle is provided, wherein the viral particle includes a modified N protein composition or a functional fragment thereof of any aforementioned aspect of the invention.

According to another aspect of the invention, a cell that includes the viral particle of any aforementioned aspect of the invention is provided.

According to another aspect of the invention, a viral immunization preparation including a viral particle of any aforementioned aspect of the invention is provided.

According to another aspect of the invention, a polynucleotide (DNA) molecule encoding a modified N protein composition of any aforementioned aspect of the invention is provided.

According to another aspect of the invention, a cell that includes the polynucleotide of any aforementioned aspect of the invention is provided.

According to another aspect of the invention, vector including the polynucleotide of any aforementioned aspect of the invention is provided.

According to another aspect of the invention, a cell including the vector of any aforementioned aspect of the invention is provided.

According to another aspect of the invention, a viral immunization preparation including the polynucleotide (DNA) molecule of any aforementioned aspect of the invention is provided.

According to another aspect of the invention, an mRNA molecule that when transcribed produces a modified N protein composition of any aforementioned aspect of the invention is provided.

According to another aspect of the invention, a cell including the mRNA molecule of any aforementioned aspect of the invention is provided.

According to another aspect of the invention, a viral immunization preparation including the mRNA molecule of any aforementioned aspect of the invention is provided.

According to another aspect of the invention, a method of modulating a subject's response to a viral exposure is provided, the method including increasing a level of a viral nucleocapsid-nucleocapsid (N-N) protein dimer in a subject exposed to or at risk of exposure to the virus, to a level effective to inhibit the viral infection in the subject compared to a control infection by the virus. In some embodiments, the control infection is infection in the absence of the increased level of the viral N-N protein dimer. In some embodiments, inhibiting the viral infection includes one or more of: reducing replication of the virus in the subject, reducing the subject's risk of mortality from the infection; and reducing severity of the infection in the subject. In some embodiments, increasing the level of the viral N-N protein dimer attenuates viral titer in the subject. In some embodiments, the N-N protein dimer is a coronavirus N-N protein dimer. In some embodiments, the N-N protein dimer is a Beta coronavirus N-N protein dimer. In some embodiments, the N-N protein dimer is a Severe Acute Respiratory Syndrome coronavirus 2 (SARS-COV-2) protein dimer, a Severe Acute Respiratory Syndrome coronavirus 1 (SARS-COV-1) protein dimer, or a Middle Eastern Respiratory syndrome coronavirus (MERs CoV) protein dimer. In some embodiments, the N-N protein dimer is a SARS-COV protein dimer. In some embodiments, the viral N proteins in the N-N protein dimer each comprise the amino acid sequence SEQ ID NO: 1 or a functional variant of SEQ ID NO: 1, and an amino acid substitution (also referred to herein as a point mutation) in the linker region of the viral N protein. In some embodiments, the amino acid substitution is a glycine→cysteine (G→C) substitution or an arginine→cystine (R→C) substitution in the viral N protein linker region. In some embodiments, the amino acid substitution is at or corresponds to amino acid G215 of SEQ ID NO: 1, or at a glycine in the functional variant of SEQ ID NO: 1 at a position that corresponds to the G215 in SEQ ID NO: 1. In some embodiments, the amino acid sequence of the modified N protein is SEQ ID NO: 2, or a functional variant of SEQ ID NO: 2. In some embodiments, the amino acid substitution is at or corresponds to amino acid G243 of SEQ ID NO: 1, or at a glycine in the functional variant of SEQ ID NO: 1 at a position that corresponds to the G243 in SEQ ID NO: 1. In some embodiments, the amino acid sequence of the modified N protein is SEQ ID NO: 3, or a functional variant of SEQ ID NO: 3. In some embodiments, the amino acid substitution is at amino acid R185 of SEQ ID NO: 1, or at an arginine in the functional variant of SEQ ID NO: 1 at a position that corresponds to the R185 in SEQ ID NO: 1. In some embodiments, the amino acid sequence of the modified N protein is SEQ ID NO: 4, or a functional variant of SEQ ID NO: 4. In some embodiments, the modified N protein has the amino acid sequence of SEQ ID NO: 5 or a functional variant of SEQ ID NO: 5. In some embodiments, the modified N protein has the amino acid sequence of SEQ ID NO: 6 or a functional variant of SEQ ID NO: 6. In some embodiments, the modified N protein has the amino acid sequence of SEQ ID NO: 7 or a functional variant of SEQ ID NO: 7. In some embodiments, the modified N protein has the amino acid sequence of SEQ ID NO: 8 or a functional variant of SEQ ID NO: 8.

### BRIEF DESCRIPTION OF THE DRAWINGS

FIG. 1A-C provides alignments, a schematic diagram, and sequences pertaining to introduction of novel cysteine residues into the SARS-Co-2 nucleocapsid linker. FIG. 1A shows alignments indicating the amino acid identity of position 215 in the SARS-COV-2 nucleocapsid protein in genomic surveillance data from 7,342,041 sequences in The International Nucleotide Sequence Database Collaboration is shown. Sequences were visualized by Taxonium11 on Jan. 10, 2024, at position 215 of the N protein. The tree was rooted to Wuhan/Hu-1 (GenBank MN908947.3, RefSeq NC_045512.2), sequences were added to the tree through the use of UShER12 and Pangolin lineage identity is labelled (B.1617.2 branch shows Delta sequences and B.1.1.529 shows Omicron sequences). FIG. 1B is a schematic diagram of the SARS-COV-2 N protein, which includes an unstructured N-terminus (residues 1-45), an N-terminal RNA binding domain (NTD; residues 45-176; in violet, PDB code 6YI3 [Dinesh, D. C. et al. PLOS Pathog. 16, e1009100 (2020)], an unstructured linker (LKR) region (176-263) which binds to Nsp3, a C-terminal dimerization domain (CTD; residues 263-365, PDB code: 6WZO [Ye, Q., et al., Protein Sci. 29, 1890-1901 (2020)], followed by an unstructured C-terminal region (residues 265-419). FIG. 1C provides sequences of the nucleocapsid linker region from SARS-COV-1 (WA-1), the Beta, Delta, and Iota isolates described herein, as well as SARS-COV and MERS. The sequences aligned using Clustal Omega. Arrows highlight positions of cysteines in the schematic shown in FIG. 1B. Figure created with Biorender. Sequences in box in FIG. 1C, top to bottom are SEQ ID NOs: 38-43.

FIG. 2A-B provides a western blot image and a graph illustrating a disulfide-bonded nucleocapsid dimer formed in several Covid variants. VeroE6-TMPRSS2 cells were infected with the indicated SARS-COV-2 variants (or WA-1, termed Wildtype) at an MOI of 0.01 for 24 hours. Unreduced cell lysates were visualized by SDS-PAGE and western blot using antibodies recognizing SARS-COV-2 N. The presence of a ˜100 kDa band recognized by the SARS-COV-2 N antibody in the Beta, Delta and Iota variants is indicated by a *. FIG. 2A shows a representative gel and FIG. 2B is a graph showing relative levels of N dimer to monomer from three independent biological replicates Mean+/−SEM is plotted, individual data points for each experiment are superimposed onto bar graphs for each condition.

FIG. 3A-D provides western blot images and graphs illustrating that N-linker cysteine residues are necessary and sufficient for stable dimer formation. FIG. 3A shows results of experiment in which HEK-293T cells were transfected with plasmids encoding the N protein of the indicated SARS-COV-2 variant. In addition, cells were transfected with constructs in which each cysteine was changed back to the parental residue in the WT (WA-1) sequence. 24 hours post transfection, unreduced cell lysates were visualized by SDS-PAGE and western blot using antibodies recognizing SARS-COV-2 N and β-actin. FIG. 3B is a graph showing the ratio of N monomer to dimer bands seen when the western blot shown in FIG. 3A was quantified. FIG. 3C is a is a western blot of results of experiment in which HEK-293T cells were transfected with plasmids encoding the WT N protein or constructs introducing a cysteine at positions 185, 215 or 243 and FIG. 3D is a graph showing the ratio of monomer to dimer bands when the western blot shown in FIG. 3C was quantified. The presence of a ˜100 kDa band recognized by the SARS-COV-2 N antibody corresponding to the stable dimer is indicated by a *. A representative western from four (FIG. 3A-B) or three (FIG. 3C-D) independent biological experiments is shown, probed for N and beta-actin. Mean+/−SEM is plotted, individual data points for each experiment are superimposed onto bar graphs for each condition.

FIG. 4A-E provides a schematic, gel images, and graphs illustrating results of studies in which the introduction of N: G215C in a WA-1 infectious clone recapitulated stable N dimer formation and displayed altered growth kinetics in HBECs. FIG. 4A is a schematic of the SARS-COV-2 genome encoding the mNG infectious clone system is shown, including the replacement of ORF7a with mNeon Green, and the introduction of the G215C mutation in N. FIG. 4B shows results from experiments in which VeroE6-TMPRSS2 cells were infected, or mock infected, with the WT or N: G215C viruses at an MOI of 0.0005 for 1 hour. 72 hpi, unreduced cell lysates were collected and visualized by SDS-PAGE and western blot using a rabbit anti-SARS-COV-2 N and beta-actin. The presence of the higher MW (˜100 kDa) band seen in the N: G215C virus is indicated with a *. A representative gel from three independent biological experiments is shown. FIG. 4C is a graph illustrating results when viral supernatants were collected at 8, 24, 32, 48, and 72 hours post infection and titered by focus forming assay (FFU; focus forming unit). FIG. 4D shows results from experiments in which human bronchial epithelial cells were infected with WT or N: G215C viruses at an MOI of 0.5 for 1 hour. In the study, unreduced cell lysates were collected and processed for western blot as in FIG. 4B. FIG. 4E shows graph of results when viral supernatants were collected sequentially from the same well at 8, 24, 32, 48, and 72 hours post infection and titered by focus forming assay (FFU; focus forming unit). Mean+/−SEM is plotted [* (p<0.05), ** (p<0.01)], N=6 from three biological experiments (FIG. 4C), N=12 from four biological experiments (FIG. 4E) is shown. Limit of detection in 20 FFU/ml.

FIG. 5A-F provides schematics, graphs, and photomicrographic images show results of studies that demonstrated that the N: G215C mutation increased viral growth in nasal washes and the lungs of Syrian golden hamsters as well as immune infiltration and damage. FIG. 5A is a schematic diagram illustrating three- to four-week old male hamsters that were intranasally infected with PBS alone or 10⁴ FFU of WT or N: G215C m Neon Green (NG) SARS-COV-2. FIG. 5B is a graph of weight-loss of infected animals that was monitored daily for 7 days post infection. On days 2 and 4 post infection, titer in the nasal wash (FIG. 5C) and right lung (FIG. 5D) were determined. On days 4 and 7 post infection, left lung tissue was harvested, fixed, cut into 5 μM section, stained with hemoxylin and eosin, scored for pathological severity (FIG. 5E) and imaged (FIG. 5F). For weights, graphs represent mean weight change+/−SEM. For viral titers, lines represent mean viral titer+/−SD. Statistical differences were determined by student's T-test with * (p<0.05), ** p<0.01).

FIG. 6A-F provides graphs and a gel image of results obtained with experiments that demonstrated that stably dimerized N was found preferentially in virions and the G215C mutation resulted in increased viral packaging of N. FIG. 6A is a graph of results obtained when HEK-293T cells were transfected with plasmids encoding the WT or G215C N protein. Gif. 6B is a graph of results obtained when Vero-TMPRSS2 cells were infected with the WT or N: G215C viruses at an MOI of 0.0005 for 1 hour. FIG. 6C is a graph of results obtained when high titer viral stocks of the WT or N: G215C viruses were concentrated by binding to 10% polyethylene glycol (PEG) then centrifugated at 10,000 G for 30 minutes at 4° C. Unreduced lysates were collected 24 hours post transfection, 72 hours post infection or directly from the concentrated viral pellet, visualized by SDS-PAGE and the ratio of N monomer to dimer was quantified. FIG. 6D is a representative western of results from FIG. 6C. the blot was probed for N and the membrane-associated M protein (M). FIG. 6E is a graph in which the ratio of N to M (normalized to the WT N: M ratio for each replicate) is shown, as well as FIG. 6F, which is a graph in which the ratio of N to Focus Forming Unit (FFU) for each stock (again normalized to the N: FFU ratio in WT virus for each replicate). N=3 for FIG. 6A-C, N=4 for FIG. 6D-F. Mean +/−SEM is plotted, individual data points for each experiment are superimposed onto bar graphs for each condition [. (p=. 05-. 1), * (p<0.05), ** (p<0.01), *** (p<. 001) or **** (p<. 0001).].

FIG. 7A-B provides microscopic images and a graph of results shown that N: G215C virions were enlarged and that they showed over-incorporation of N. FIG. 7A shows images of results of experiments in which Vero-TMPRSS2 cells were infected with WT or N: G215C viruses at an MOI of 0.1. The following day cells were prepared for electron microscopy by high-pressure freezing and freeze-substitution, then sectioned and imaged by dual-axis electron tomography. Virus-containing exit compartments were located in both samples, and virions that had completely separated from cellular membranes were selected and analyzed in 3D in order to determine the structure of intact virions and the arrangement of internal ribonucleoprotein complexes. FIG. 7B is a graph showing measurements of the maximum diameter of 20 randomly selected virions for each virus. Mean+/−SEM is plotted, individual data points for each experiment are superimposed onto bar graphs for each condition, **** (p<. 0001).

FIG. 8: provides schematics illustrating that mutations in the nucleocapsid linker may alter the oligomerization status. The SARS-COV-2 N protein is composed of RNA-binding and dimerization domains, interspersed with flexible unstructured regions at the N and C-termini and a linker region in the middle of the protein. Mutations at three separate sites in the central linker region introduce novel cysteines that differentially increase dimerization levels via a disulfide bond. (RNA-binding: PDB code 6YI3 [Dinesh, D. C. et al. PLOS Pathog. 16, e1009100 (2020)], Dimerization Domain PDB code: 6WZO [Ye, Q., et al., Protein Sci. 29, 1890-1901 (2020)]). Results of studies described herein suggest these interactions occur between the linker regions of pairs of N-N dimers and mediate different levels of higher order N-N oligomerization. This figure was made using BioRender.

FIG. 9 provides alignments of sequences and illustrates the absence of cysteines in the linker region of Coronaviridae nucleocapsid proteins. Sequences for the indicated coronavirus nucleocapsid protein sequences were obtained through the NIH Protein Databank and aligned using Clustal Omega from EMBL-EBI (Clustal 0 (1.2.4)). The box highlights the linker region (residues 175-247 in SARS-COV-2). The only cysteine in the displayed sequences occurs immediately before the linker region in Erinaceus betacoronavirus. The sequences shown in FIG. 9 from top (NL63) to bottom (Bat Coronavirus) are SEQ ID NOs: 44-61.

FIG. 10A-G provides gel images and a table illustrating results of studies that demonstrated SARS-COV-2 N dimer formation. In the studies, VeroE6-TMPRSS2 cells were infected, or mock infected, with the indicated SARS-COV-2 variants (or WA-1, termed Wildtype) at an MOI of 0.01 for 24 hours. Cell lysates were harvested under (FIG. 10A) reducing conditions (10 mM DTT) or (FIG. 10B) non-reducing conditions, but in the presence of NEM (which prevents the formation of post-lysis disulfide bonds) before visualization by SDS-PAGE and western blot using antibodies recognizing SARS-COV-2 N and β-actin. Alternatively, unreduced lysates from cells infected as above were visualized by SDS-PAGE and western blot using two different antibodies recognizing SARS-COV-2 N: (FIG. 10C) Invitrogen anti-N mouse antibody (MA5-35943, in green) and (FIG. 10D) Sino Biological anti-N rabbit antibody (40143-R001). FIG. 10E shows results when lysates were stained and imaged simultaneously with the two anti-N antibodies and the overlay is shown. Note minor bands that are seen with the Invitrogen but not the Sino-Biological antibody. The MW of the ˜100 kDa band observed in Beta, Delta and Iota samples under non-reducing conditions is indicated by a *. A representative gel from 3 (FIG. 10A-B) or 2 (FIG. 10C) independent biological replicates is shown. FIG. 10F shows results of experiment in which Vero-TMPRSS2 cells were infected with WA1 or Delta SARS-COV-2 at an MOI of 0.01. 24 hpi cells were harvested, lysed and N was affinity purified using immunoprecipitation. N and associated proteins were run on an SDS-PAGE gel, and bands corresponding to the monomer and dimer were cut and processed for mass spectrometry. FIG. 10G is a table showing the number of peptides for each SARS-COV-2 viral protein found in the monomer or dimer portion of the gel, for either WT (WA-1) or Delta.

FIG. 11A-B provides graphs and photomicrographic images of studies performed to assess WT and N: G215C antigen distribution. FIG. 11A provides graphs of antigen scores in Parenchyma, Airway, and Overall, on days 2, 4, and 7. FIG. 11B provides images of WT and N: G215C on days 2, 4, and 7. At day 2, both WT and N: G215C had similar antigen distribution and scores.

Brief Description of Sequences
SEQ ID NO: 1 Amino acid sequence of SARS-CoV-2 N
protein (WA-1):
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNGGDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDFSKQLQQSMSSADSTQA.
SEQ ID NO: 2 Amino acid sequence of SARS-CoV-2 Delta
variant N protein:
MSDNGPQNQRNAPRITFGGPSDSTGSNQNGERSGARSKQRRPQGLPNNTASWFTA
LTQHGKEGLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEgsrggsqassrsssrsrnssrnstpgssmgTSPARMAGNGCDaala
llllDRLNQLESKMSgkgqqqqgqtvtkkSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPkkdkkkkAYETQALPQR
QKKQQTVTLLPAADLDDFSKQLQQSMSSADSTQA.
SEQ ID NO: 3 Amino acid sequence of SARS-CoV-2 Beta
variant N protein:
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEgsrggsqassCsssrsrnssrnstpgssrgISPARMagnggdaala
lllldrlnqIESKMSgkgqqqqgqtvtkkSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYktfpptcpkkdkkkkadctQALPQR
QKKQQTVTLLPAADLDDFSKQLQQSMSSADSTQA.
SEQ ID NO: 4 Amino acid sequence of SARS-CoV-2 Iota
variant N protein:
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTLGSSRGTSPARMAGNGGDAALA
LLLLDRLNQLESKISGKGQQQQCQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDESKQLQQSMSSADSTQA.
SEQ ID NO: 5 Sequence of SARS-CoV-2 N protein with R185C
substitution (WA-1 R185C):
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSCSSSRSRNSSRNSTPGSSRGTSPARMAGNGGDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDFSKQLQQSMSSADSTQA.
SEQ ID NO: 6 Sequence of SARS-CoV-2 N protein with G215C
substitution (WA-1 G215C):
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNGCDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDFSKQLQQSMSSADSTQA.
SEQ ID NO: 7 Sequence of SARS-CoV-2 N protein with G243C
substitution (WA-1 G243C):
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNGGDAALA
LLLLDRLNQLESKMSGKGQQQQCQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDFSKQLQQSMSSADSTQA.
SEQ ID NO: 8 Sequence of SARS-CoV-2 N protein with G214C
substitution (WA-1 G214C):
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNCGDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQR
QKKQQTVTLLPAADLDDESKQLQQSMSSADSTQA.
SEQ ID NO: 9 Sequence of SARS-CoV-2 WA-1 linker domain
amino acid sequence:
GSRGGSQASSRSSSRSRNSSRNSTPGSSRGTSPARMAGNGGDAALALLLLDRLNQ
LESKMSGKGQQQQGQTVTK.
SEQ ID NO: 10 DNA sequence encoding linker domain
(SEQ ID NO: 9):
gggagcagaggcggcagtcaagcctcttctcgttcctcatcacgtagtcgcaaca
gttcaagaaattcaactccaggcagcagtaggggaacttctcctgctagaatggc
tggcaatggcggtgatgctgctcttgctttgctgctgcttgacagattgaaccag
cttgagagcaaaatgtctggtaaaggccaacaacaacaaggccaaactgtcacta
ag.
SEQ ID NO: 11 amino acid sequence of SARS-CoV-2 Delta
variant N protein linker domain:
GSRGGSQASSRSSSRSRNSSRNSTPGSSMGTSPARMAGNGCDAALALLLLDRLNQ
LESKMSGKGQQQQGQTVTK.
SEQ ID NO: 12 DNA sequence encoding linker domain
(SEQ ID NO: 11):
gggagcagaggcggcagtcaagcctcttctcgttcctcatcacgtagtcgcaaca
gttcaagaaattcaactccaggcagcagtatgggaacttctcctgctagaatggc
tggcaatggctgtgatgctgctcttgctttgctgctgcttgacagattgaaccag
cttgagagcaaaatgtctggtaaaggccaacaacaacaaggccaaactgtcacta
ag.
SEQ ID NO: 13 amino acid sequence of SARS-CoV-2 Beta
variant N protein linker domain
GSRGGSQASSCSSSRSRNSSRNSTPGSSRGISPARMAGNGGDAALALLLLDRLNQ
LESKMSGKGQQQQGQTVTK.
SEQ ID NO: 14 DNA sequence encoding linker domain
(SEQ ID NO: 13):
gggagcagaggcggcagtcaagcctcttcttgttcctcatcacgtagtcgcaaca
gttcaagaaattcaactccaggcagcagtaggggaatttctcctgctagaatggc
tggcaatggcggtgatgctgctcttgctttgctgctgcttgacagattgaaccag
cttgagagcaaaatgtctggtaaaggccaacaacaacaaggccaaactgtcacta
ag.
SEQ ID NO: 15 amino acid sequence of SARS-CoV-2 Iota
variant N protein linker domain
SRGGSQASSRSSSRSRNSSRNSTLGSSRGTSPARMAGNGGDAALALLLLDRLNQL
ESKISGKGQQQQCQTVTK
SEQ ID NO: 16 DNA sequence encoding linker domain
(SEQ ID NO: 15):
agcagaggcggcagtcaagcctcttctcgttcctcatcacgtagtcgcaacagtt
caagaaattcaactctaggcagcagtaggggaacttctcctgctagaatggctgg
caatggcggtgatgctgctcttgctttgctgctgcttgacagattgaaccagctt
gagagcaaaatatctggtaaaggccaacaacaacaatgccaaactgtcactaag.
SEQ ID NO: 17 DNA sequence encoding SARS-CoV-2 N
protein (WA-1:)
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactccaggcagcag
taggggaacttctcctgctagaatggctggcaatggcggtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 18 DNA sequence encoding SARS-CoV-2 Delta
variant N protein:
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaaggccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctatgcagaagggagcagaggcggcagtcaagcctctt
ctcgttcctcatcacgtagtcgcaacagttcaagaaattcaactccaggcagcag
tatgggaacttctcctgctagaatggctggcaatggctgtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcgacaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggcttatgaaactcaagccttaccgcagaga
cagaagaaacagcaaactgtgactcttcttcctgctgcagatttggatgatttct
ccaaacaattgcaacaatccatgagtagtgctgactcaactcaggcc.
SEQ ID NO: 19 DNA sequence encoding SARS-CoV-2 Beta
variant N protein:
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
cttgttcctcatcacgtagtcgcaacagttcaagaaattcaactccaggcagcag
taggggaatttctcctgctagaatggctggcaatggcggtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 20 DNA sequence encoding SARS-CoV-2 Iota
variant N protein:
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactctaggcagcag
taggggaacttctcctgctagaatggctggcaatggcggtgatgctgctcttgct
ttgctgctgcttgacagattgaaccagcttgagagcaaaatatctggtaaaggcc
aacaacaacaatgccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 21 DNA sequence encoding SARS-CoV-2 N
protein with R185C substitution
(WA-1 R185C):
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
cttgttcctcatcacgtagtcgcaacagttcaagaaattcaactccaggcagcag
taggggaacttctcctgctagaatggctggcaatggcggtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 22 DNA sequence encoding SARS-CoV-2 N
protein with G215C substitution
(WA-1 G215C):
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactccaggcagcag
taggggaacttctcctgctagaatggctggcaatggctgtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 23 DNA sequence encoding SARS-CoV-2 N
protein with G243C substitution
(WA-1 G243C):
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactccaggcagcag
taggggaacttctcctgctagaatggctggcaatggcggtgatgctgctcttgct
ttgctgctgcttgacagattgaaccagcttgagagcaaaatgtctggtaaaggcc
aacaacaacaatgtcaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcc.
SEQ ID NO: 24 DNA sequence encoding SARS-CoV-2 N
protein with G214C substitution
(WA-1 G214C):
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactccaggcagcag
taggggaacttctcctgctagaatggctggcaattgcggtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattcccaccaacaga
gcctaaaaaggacaaaaagaagaaggctgatgaaactcaagccttaccgcagaga
cagaagaaacagcaaactgtgactcttcttcctgctgcagatttggatgatttct
ccaaacaattgcaacaatccatgagcagtgctgactcaactcaggcctaa.
SEQ ID NO: 25 Amino acid sequence of SARS-CoV-2 linker
domain plus dimerization domain:
GSRGGSQASSRSSSRSRNSSRNSTPGSSRGTSPARMAGNGGDAALALLLLDRLNQ
LESKMSGKGQQQQGQTVTKKSAAEASKKPRQKRTATKAYNVTQAFGRRGPEQTQG
NFGDQELIRQGTDYKHWPQIAQFAPSASAFFGMSRIGMEVTPSGTWLTYTGAIKL
DDKDPNFKDQVILLNKHIDAYKTFP.
SEQ ID NO: 26 DNA sequence encoding SARS-CoV-2 linker
domain plus dimerization domain
(SEQ ID NO: 25):
gggagcagaggcggcagtcaagcctcttctcgttcctcatcacgtagtcgcaaca
gttcaagaaattcaactccaggcagcagtaggggaacttctcctgctagaatggc
tggcaatggcggtgatgctgctcttgctttgctgctgcttgacagattgaaccag
cttgagagcaaaatgtctggtaaaggccaacaacaacaaggccaaactgtcacta
agaaatctgctgctgaggcttctaagaagcctcggcaaaaacgtactgccactaa
agcatacaatgtaacacaagctttcggcagacgtggtccagaacaaacccaagga
aattttggggaccaggaactaatcagacaaggaactgattacaaacattggccgc
aaattgcacaatttgcccccagcgcttcagcgttcttcggaatgtcgcgcattgg
catggaagtcacaccttcgggaacgtggttgacctacacaggtgccatcaaattg
gatgacaaagatccaaatttcaaagatcaagtcattttgctgaataagcatattg
acgcatacaaaacattccca.
SEQ ID NO: 27 is amino acid sequence of SARS-CoV-2 N
protein without N-terminal linker:
PNNTASWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRNPANNA
AIVLQLPQGTTLPKGFYAEGSRGGSQASSRSSSRSRNSSRNSTPGSSRGTSPARM
AGNGGDAALALLLLDRLNQLESKMSGKGQQQQGQTVTKKSAAEASKKPRQKRTAT
KAYNVTQAFGRRGPEQTQGNFGDQELIRQGTDYKHWPQIAQFAPSASAFFGMSRI
GMEVTPSGTWLTYTGAIKLDDKDPNFKDQVILLNKHIDAYKTFPPTEPKKDKKKK
ADETQALPQRQKKQQTVTLLPAADLDDESKQLQQSMSSADSTQA.
SEQ ID NO: 28 is DNA sequence encoding SARS-CoV-2 N
protein without N-terminal linker
(SEQ ID NO: 27):
cccaataatactgcgtcttggttcaccgctctcactcaacatggcaaggaagacc
ttaaattccctcgaggacaaggcgttccaattaacaccaatagcagtccagatga
ccaaattggctactaccgaagagctaccagacgaattcgtggtggtgacggtaaa
atgaaagatctcagtccaagatggtatttctactacctaggaactgggccagaag
ctggacttccctatggtgctaacaaagacggcatcatatgggttgcaactgaggg
agccttgaatacaccaaaagatcacattggcacccgcaatcctgctaacaatgct
gcaatcgtgctacaacttcctcaaggaacaacattgccaaaaggcttctacgcag
aagggagcagaggcggcagtcaagcctcttctcgttcctcatcacgtagtcgcaa
cagttcaagaaattcaactccaggcagcagtaggggaacttctcctgctagaatg
gctggcaatggcggtgatgctgctcttgctttgctgctgcttgacagattgaacc
agcttgagagcaaaatgtctggtaaaggccaacaacaacaaggccaaactgtcac
taagaaatctgctgctgaggcttctaagaagcctcggcaaaaacgtactgccact
aaagcatacaatgtaacacaagctttcggcagacgtggtccagaacaaacccaag
gaaattttggggaccaggaactaatcagacaaggaactgattacaaacattggcc
gcaaattgcacaatttgcccccagcgcttcagcgttcttcggaatgtcgcgcatt
ggcatggaagtcacaccttcgggaacgtggttgacctacacaggtgccatcaaat
tggatgacaaagatccaaatttcaaagatcaagtcattttgctgaataagcatat
tgacgcatacaaaacattcccaccaacagagcctaaaaaggacaaaaagaagaag
gctgatgaaactcaagccttaccgcagagacagaagaaacagcaaactgtgactc
ttcttcctgctgcagatttggatgatttctccaaacaattgcaacaatccatgag
cagtgctgactcaactcaggcctaa.
SEQ ID NO: 29 is amino acid sequence of SARS-CoV-2 N
protein without C-terminal linker:
MSDNGPQNQRNAPRITFGGPSDSTGSNQNGERSGARSKQRRPQGLPNNTASWFTA
LTQHGKEDLKFPRGQGVPINTNSSPDDQIGYYRRATRRIRGGDGKMKDLSPRWYF
YYLGTGPEAGLPYGANKDGIIWVATEGALNTPKDHIGTRNPANNAAIVLQLPQGT
TLPKGFYAEGSRGGSQASSRSSSRSRNSSRNSTPGSSRGTSPARMAGNGGDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDA YKTFP.
SEQ ID NO: 30 is DNA sequence encoding SARS-CoV-2 N
protein without C-terminal linker
(SEQ ID NO: 29):
atgtctgataatggaccccaaaatcagcgaaatgcaccccgcattacgtttggtg
gaccctcagattcaactggcagtaaccagaatggagaacgcagtggggcgcgatc
aaaacaacgtcggccccaaggtttacccaataatactgcgtcttggttcaccgct
ctcactcaacatggcaaggaagaccttaaattccctcgaggacaaggcgttccaa
ttaacaccaatagcagtccagatgaccaaattggctactaccgaagagctaccag
acgaattcgtggtggtgacggtaaaatgaaagatctcagtccaagatggtatttc
tactacctaggaactgggccagaagctggacttccctatggtgctaacaaagacg
gcatcatatgggttgcaactgagggagccttgaatacaccaaaagatcacattgg
cacccgcaatcctgctaacaatgctgcaatcgtgctacaacttcctcaaggaaca
acattgccaaaaggcttctacgcagaagggagcagaggcggcagtcaagcctctt
ctcgttcctcatcacgtagtcgcaacagttcaagaaattcaactccaggcagcag
taggggaacttctcctgctagaatggctggcaatggcggtgatgctgctcttgct
ttgctgctgcttgacagattgaaccagcttgagagcaaaatgtctggtaaaggcc
aacaacaacaaggccaaactgtcactaagaaatctgctgctgaggcttctaagaa
gcctcggcaaaaacgtactgccactaaagcatacaatgtaacacaagctttcggc
agacgtggtccagaacaaacccaaggaaattttggggaccaggaactaatcagac
aaggaactgattacaaacattggccgcaaattgcacaatttgcccccagcgcttc
agcgttcttcggaatgtcgcgcattggcatggaagtcacaccttcgggaacgtgg
ttgacctacacaggtgccatcaaattggatgacaaagatccaaatttcaaagatc
aagtcattttgctgaataagcatattgacgcatacaaaacattccca.
SEQ ID NO: 31 is a non-limiting example of a fragment of
SEQ ID NO: 2.
SRGGSQASSRSSSRSRNSSRNSTPGSSMGTSPARMAGNGCDAALALLLLDRLNQL
ESKMSGKGQQQQGQTVTKKSAAEASKKPRQKRTATKAYNVTQAFGRRGPEQTQGN
FGDQELIRQGTDYKHWPQIAQFAPSASAFFGMSRIGMEVTPSGTWLTYTGAIKLD
DKDPNFKDQVILLNKHIDAYKTFPP.
SEQ ID NO: 32 is an example of a fragment of SEQ ID NO: 3.
GDGKMKDLSPRWYFYYLGTGPEAGLPYGANKDGIIWVATEGALNTPKDHIGTRNP
ANNAAIVLQLPQGTTLPKGFYAEGSRGGSQASSCSSSRSRNSSRNSTPGSSRGIS
PARMAGNGGDAALALLLLDRLNQLESKMSGKGQQQQGQTVTKKSAAEASKKPRQK
RTATKAYNVTQAFGRRGPEQTQGNFGDQELIRQGTDYKHWPQIAQFAPSASAFFG
MSRIGMEVTPSGTWLTYTGAIKLDDKDPNFKDQVILLNKHIDAYKTFPP.
SEQ ID NO: 33 is an example of a fragment of SEQ ID NO: 4:
SRGGSQASSRSSSRSRNSSRNSTLGSSRGTSPARMAGNGGDAALALLLLDRLNQL
ESKISGKGQQQQCQTVTKKSAAEASKKPRQKRTATKAYNVTQAFGRRGPEQTQGN
FGDQELIRQGTDYKHWPQIAQFAPSASAFFGMSRIGMEVTPSGTWLTYTGAIKLD
DKDPNFKDQVILLNKHIDAYKTFPPTEPKKDKKKKADETQALPQRQKKQ.
SEQ ID NO: 34 is an example of a fragment of SEQ ID NO: 5:
PANNAAIVLQLPQGTTLPKGFYAEGSRGGSQASSCSSSRSRNSSRNSTPGSSRGT
SPARMAGNGGDAALALLLLDRLNQLESKMSGKGQQQQGQTVTKKSAAEASKKPRQ
KRTATKAYNVTQAFGRRGPEQTQGNFGDQELIRQGTDYKHWPQIAQFAPSASAFF
GMSRIGMEVTPSGTWLTYTGAIKLDDKDPNFKDQVILLNKHIDAYKTFPP.
SEQ ID NO: 35 is an example of a fragment of SEQ ID NO: 6:
SRGGSQASSRSSSRSRNSSRNSTPGSSRGTSPARMAGNGCDAALALLLLDRLNQL
ESKMSGKGQQQQGQTVTKKSAAEASKKPRQKRTATKAYNVTQAFGRRGPEQTQGN
FGDQELIRQGTDYKHWPQIAQFAPSASAFFGMSRIGMEVTPSGTWLTYTGAIKLD
DKDPNFKDQVILLNKHIDAYKTFPPTEP.
SEQ ID NO: 36 is an example of a fragment of SEQ ID NO: 7:
NQNGERSGARSKQRRPQGLPNNTASWFTALTQHGKEDLKFPRGQGVPINTNSSPD
DQIGYYRRATRRIRGGDGKMKDLSPRWYFYYLGTGPEAGLPYGANKDGIIWVATE
GALNTPKDHIGTRNPANNAAIVLQLPQGTTLPKGFYAEGSRGGSQASSRSSSRSR
NSSRNSTPGSSRGTSPARMAGNGGDAALALLLLDRLNQLESKMSGKGQQQQCQTV
TKKSAAEASKKPRQKRTATKAYNVTQAFGRRGPEQTQGNFGDQELIRQGTDYKHW
PQIAQFAPSASAFFGMSRIGMEVTPSGTWLTYTGAIKLDDKDPNFKDQVILLNKH
IDAYKTFPPTEPKKDKKKKADETQALPQRQKKQQTVTLLPA.
SEQ ID NO: 37 is an example of a fragment of SEQ ID NO: 8:
SRGGSQASSRSSSRSRNSSRNSTPGSSRGTSPARMAGNCGDAALALLLLDRLNQL
ESKMSGKGQQQQGQTVTKKSAAEASKKPRQKRTATKAYNVTQAFGRRGPEQTQGN
FGDQELIRQGTDYKHWPQIAQFAPSASAFFGMSRIGMEVTPSGTWLTYTGAIKLD
DKDPNFKDQVILLNKHIDAYKTFPP.

### DETAILED DESCRIPTION

The evolution of SARS-COV-2 variants and their respective phenotypes represent an important set of tools to understand basic coronavirus biology as well as the public health implications of individual mutations in variants of concern. While mutations outside of the Spike protein are not well studied, the entire viral genome is undergoing evolutionary selection, particularly the central disordered linker region of the nucleocapsid (N) protein. Studies disclosed herein have identified amino acid substitutions that when made in the linker domain of a SARS-COV N protein, result in the formation of a disulfide bond and a stable N-N dimer. Using reverse genetics, it has now been determined that the substitutions, which result in inclusion of a cysteine residue in the substituted position, are necessary and sufficient for stable dimer formation in a WT SARS-COV-2 background, where it results in significantly increased viral growth both in vitro and in vivo. It has also now been demonstrated that a virus comprising a modified N protein of the invention packages more nucleocapsid per virion and that individual virions are larger, with elongated morphologies.

The invention, in part, includes compositions comprising a modified viral nucleocapsid (N) protein, wherein the modified viral N protein comprises an amino acid substitution (also referred to herein as a point mutation) in the linker region of the N protein. Other compositions of the invention include sequences encoding a modified viral N protein of the invention, cells comprising a modified N protein of the invention, cells comprising a molecule encoding a modified N protein of the invention, fusion proteins comprising a modified N protein of the invention, and vectors comprising a sequence encoding a modified N protein of the invention. Compositions, such as those described herein, can be used in methods of the invention, including but not limited to, increasing the antigenicity of a viral immunization preparation by including in the preparation a modified N protein of the invention, or a sequence that encodes a modified N protein of the invention. A non-limiting example methods of using such preparations of the invention is for generating anti-virus vaccines. Another non-limiting example of a method of the invention, comprises increasing a level of N-N dimers in a virus particle. It has now been discovered that N-N dimers are present in viral particles that comprise a modified N protein of the invention. Another non-limiting example of a method of the invention includes increasing a level of a viral N-N protein dimer in a subject. Increasing the level of N-N dimers in a subject who is exposed to or is at risk of exposure to the virus, can be used to inhibit the viral infection in the subject compared to a control level of infection by the virus.

In some embodiments of the invention, a modified N protein is an N protein of a SARS-CoV-2 virion comprising at least one amino acid substitution in the linker domain of the SARS-CoV-2 N protein. An example of an amino acid sequence of a wild-type SARS-COV-2 N protein is shown herein as SEQ ID NO: 1. A linker domain sequence of the SARS-COV-2 N protein is shown herein as SEQ ID NO: 9.

As described herein, it has now been discovered that substitution of one or more specific amino acids in the linker region of a virion N protein results in a modified N protein that forms dimers, which are referred to herein as N-N protein dimers, or N-N dimers. It has now been identified that the formation of N-N protein dimers greatly improves antigenicity in vaccine preparation. In some embodiments the amino acid substitution includes inserting a cysteine within the linker region of a viral N protein. In some embodiments, the substitution includes replacing an arginine in the viral N protein linker domain with a cysteine. In some embodiments a substitution includes replacing a glycine in the viral N protein linker domain with a cysteine.

It has now been discovered that introducing a cysteine residue within the viral N protein linker domain has major structural implications as it results in the production of a stable N-N dimer linked by a disulfide bond. As disclosed herein, presence of a novel cysteine within the linker region of the N protein plays a role in viral replication and particle formation. As a non-limiting example, it has been experimentally determined that a modified N protein comprising a G→C substitution at the amino acid corresponding to amino acid at position 215 in the SARS-CoV-2 N protein, resulted in substantially increased viral replication kinetics in primary differentiated human bronchial cells. The N protein G215C substitution also increased viral replication in the nasal washes and lungs of infected Syrian golden hamsters, while paradoxically delaying weight loss. Finally, it has also now been demonstrated that a virus comprising the N protein with a G→C substitution at the position corresponding to amino acid 215 in the SARS-COV-2 N protein set forth herein as SEQ ID NO: 1, packaged substantially more N per virion and many of the virions displayed elongated morphology. Together, the results and data set forth herein provide evidence that modified N proteins of the invention increase levels of N-N dimers in virions, which drives increased packaging of N into mature virions and results in significant increases in viral replication both in vitro and in vivo.

### Introduction of a Novel Cysteine in the Nucleocapsid Linker Region of Variants of Concern

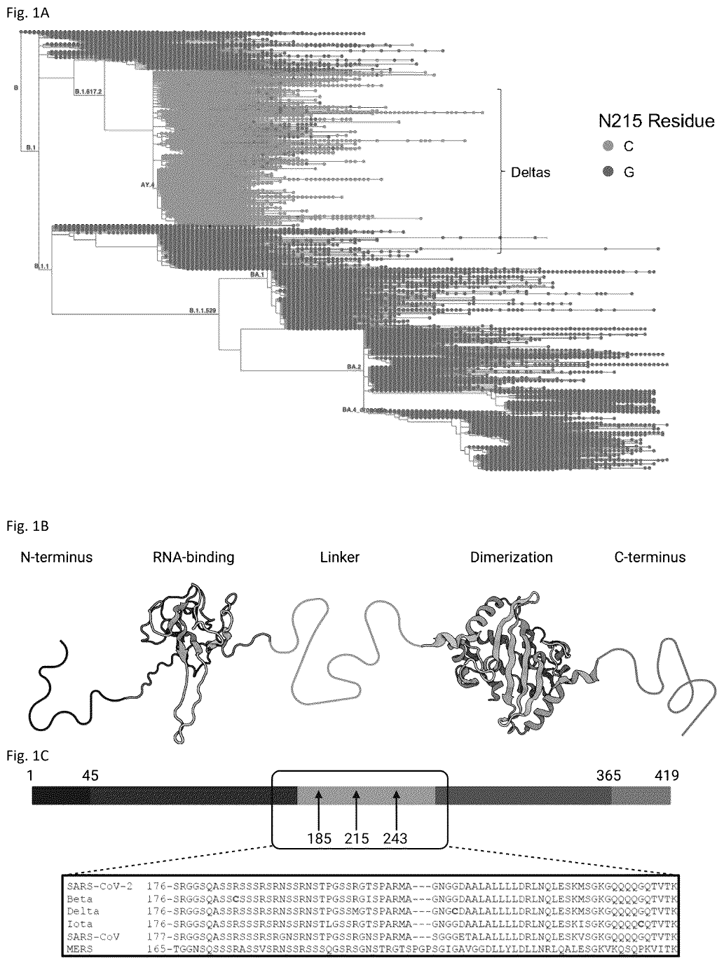

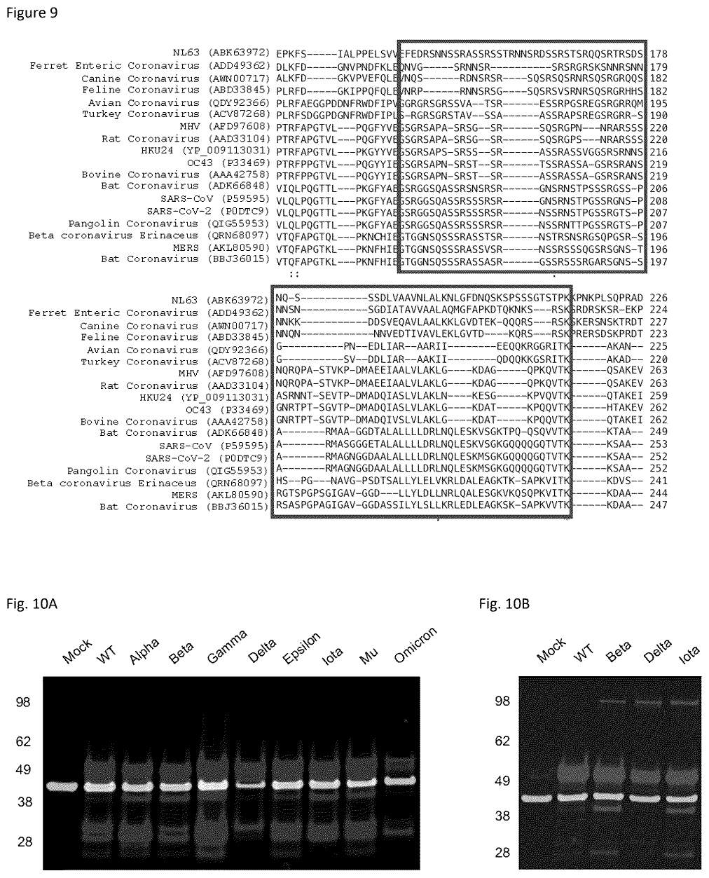

The unprecedented access to sequences of SARS-COV-2 genomes acquired from individual infected people in near real-time throughout the COVID-19 pandemic has revealed a detailed picture of the evolution of this viral pandemic over the last four years. Mutations introduced by the viral RNA-dependent RNA-polymerase led to the emergence of distinct lineages, characterized by suites of different mutations. Variants that met certain public health benchmarks, termed Variants of Concern (VOCs), contained individual mutations in viral proteins that conferred distinct evolutionary advantages. The G215C mutation described herein sits in the disordered linker region of the nucleocapsid protein, which lies between a N terminal RNA-binding domain (RBD) and a C terminal dimerization domain (FIG. 1B). The introduction of a cysteine in the SARS-COV-2 nucleocapsid is unique amongst zoonotic Betacoronavirus, as neither SARS-COV, MERS nor SARS-COV-2 nucleocapsids contained any cysteines (FIG. 1C). Furthermore, while other coronavirus nucleocapsid proteins do contain cysteines, they are largely absent from the linker region (FIG. 9). Surprisingly, when analyzing the sequences of a panel of VOCs obtained from clinical specimens, it was discovered that two other variant (Beta & Iota) isolates also contained a cysteine within the N protein (FIG. 1C). Interestingly, all three of these mutations sit within the intrinsically disordered linker region of N (also termed N3/sN3) between N2 (RNA binding domain) and N4 (dimerization domain). Both the Beta (B.1.351) and Iota (B.1.526) stocks contained novel cysteines in the linker region, R185C (99.7% of reads) and G234C (100% of reads), respectively (FIG. 1C). Because the introduction of a cysteine residue would allow for the formation of a new disulfide-bonded N-N dimer complex, experiments were performed to determine whether this mutation could have major impacts on the secondary, tertiary and/or quaternary protein structure of the nucleocapsid protein.

### Viral Infections and Symptoms

A viral infection, which may also be referred to as a viral disease, results in a cell or subject when a pathogenic virus is present in a cell or subject, or contacts a cell or subject, and infectious virus particles (virions) attach to and enter one or more cells. A viral infection in a cell, as referenced herein, means a cell into which virions have entered. A virally infected cell may be in a subject (in vivo) or obtained from a subject. In some embodiments, a virally infected cell is a cell in culture (in vitro), or is an infected cell obtained from culture. Numerous viruses are known to infect subjects and cells. Categories of infective viruses include DNA viruses and RNA viruses, including single-stranded, double-stranded, and partly double-stranded viruses. Certain types of viruses are envelope viruses, meaning they are encapsulated with a lipid membrane, which comes from an infected cell when new virus particles “bud off” from the infected cell. The lipid membrane comprises material from the infected cell's plasma membrane.

With respect to RNA viruses, positive single-stranded RNA virus families include non-enveloped viruses, such as Astroviridae, Caliciviridae and Picornaviridae; and enveloped viruses, such as Coronaviridae, Flaviviridae, Retroviridae and Togaviridae. Negative single-stranded RNA families include Arenaviridae, Bunyaviridae, Filoviridae, Orthomyxoviridae, Paramyxoviridae and Rhabdoviridae, all of which are enveloped viruses. In some embodiments of the invention, compositions and methods of the invention are applied to RNA viruses. In certain embodiments of the invention, compositions and methods of the invention are applied to an infection by a positive single-stranded RNA virus, optionally a coronaviridae infection. In some embodiments of the invention, a virus that infects a cell or subject is a SARS-COV virus, and optionally is a SARS-COV-2 virus. With respect to DNA viruses, double-stranded DNA virus families include non-enveloped viruses, such as Adenoviridae, Papovaviridae, and Poxviridae, and enveloped viruses such as Herpesviridae. Single-stranded DNA virus families include non-enveloped viruses, such as Parvoviridae and Anelloviridae. In some embodiments of the invention, compositions and methods of the invention are applied to DNA viruses.

As used herein, the term “viral particle” refers to an infectious viral particle or virion, whose main function is to deliver its genome (DNA or RNA) into a host cell so that its genome can be expressed, e.g., transcribed and translated, by the host cell. A complete viral particle includes one or more types of viral proteins (also referred to herein as “protein(s) of the virus”) and at least one complete copy of the viral genome (also referred to herein as a “polynucleotide component of the virus”). Several main types of viral proteins exist, including structural proteins, non-structural proteins, and regulatory and accessory proteins. Viral structural proteins include capsid proteins, envelope proteins, and membrane fusion proteins; viral non-structural proteins include proteins involved in replicon (replication complex) formation and immunomodulation (modulating the immune response of a subject to an infected cell). Viral regulatory and accessory proteins have a variety of functions, including but not limited to controlling viral gene expression in the host cell. The number and function(s) of each type of viral protein vary from virus to virus. In certain aspects of the invention, the viral N protein is a coronavirus N protein. In some embodiments of the invention, the viral N protein is a Beta coronavirus protein. In some embodiments of the invention, a viral protein is a nucleocapsid (N) protein, which binds to and organizes the viral genome. In some embodiments of the invention, the N protein is a coronavirus N protein or a functional fragment thereof. In some embodiments of the invention the N protein is a SARS-COV N protein or a functional fragment thereof. In some embodiments of the invention the N protein is a SARS-COV-1 N protein or a functional fragment thereof. In some embodiments of the invention the N protein is a SARS-COV-2 N protein or a functional fragment thereof.

Some aspects of the invention, include methods of modulating a cell or subject's response to a viral exposure. The term “modulating a response” as used herein with respect to a viral infection in a cell or subject means one or more of: reducing the response of a cell or subject to exposure to the virus; reducing a response to a viral exposure such that the viral replication and/or propagation is statistically significantly reduced in comparison to a control, and reducing one or more symptoms of a viral infection in a cell or subject such that the symptom or symptoms are statistically significantly ameliorated in comparison to a control. As described elsewhere herein, a control be a cell or subject that is exposed to the virus and does not comprise a modified viral N protein of the invention and/or an N-N dimer of the invention. In a non-limiting example, modulation of a cell or subject's response to a viral exposure can be determined in a subject comprising a modified N protein and/or an N-N dimer of the invention and the result compared to modulation of a control cell or subject's response to a viral exposure when that subject is treated with an existing therapy known and used in the art for the viral infection being treated or for similar types of viral infections (e.g., viruses from the same family; viruses that infect similar cell or tissue types; or viral infections that result in similar symptoms), may be a placebo, or may be no treatment at all. In some embodiments, a modulated response to a viral exposure may be a statistically significant reduction in viral replication in a cell or subject that is at least a 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99%, or 100% reduction compared a control level of viral replication, including all percentages within that range. In some embodiments, a modulated response to a viral exposure may be a statistically significant reduction in symptoms in the cell or subject that is at least a 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99%, or 100% reduction compared to a control treatment, including all percentages within that range.

A change in viral replication can be determined using a method of detecting an amount of viral particles, for example in a sample obtained from a subject, and comparing that determined amount to a control amount. In some embodiments of the invention, viral replication may be detected using molecular detection methods. Molecular detection methods are routine practices in the art and a skilled artisan would be able to use such methods in conjunction with the teachings provided herein. Non-limiting examples of molecular detection methods include PCR-based methods (e.g., endpoint PCR, quantitative PCR (qPCR), real-time PCR (rtPCR), and reverse-transcriptase PCR (RT-PCR)), CRISPR-based methods, and immunological methods (e.g., ELISA). In some aspects, modulating a response to exposure to a virus also refers to reducing a viral infection such that viral replication is reduced to levels that are undetectable by molecular detection methods, though one skilled in the art will understand that suppressing a viral infection may not involve eradicating all viral particles.

The term “modulating a response to a viral exposure” in a cell or subject may also be used herein in reference to reducing one or more symptoms of the viral infection in the cell or subject so the one or more symptoms are statistically significantly ameliorated in comparison to the one or more symptoms in a control cell or subject. A statistically significant amelioration of one or more symptoms of a viral infection in a cell or subject may be an at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99%, or 100% reduction compared to a control, including all percentages within that range.

As used herein, the term “amelioration” refers to improvement in severity of one or more symptoms of a viral infection in a cell or subject compared to a control severity or compared to the severity of the one or more symptoms determined in a cell or subject at an earlier time point in their viral infection. Non-limiting examples of amelioration also include a reduction in number and/or severity of one or more symptoms of the viral infection in a cell or subject exposed to the virus, and a reduction in overall duration of symptoms in a cell or subject exposed to the virus, and a reduction in viral load in a cell or subject exposed to the virus. Amelioration of viral infection symptoms may be evaluated and/or measured using art-known methods that will be familiar to those with ordinary skill in the art. In some aspects, modulating a response to a viral exposure in a cell or subject may refer to treating infection by the virus such that one or more symptoms of the viral infection in a cell or subject are eliminated or apparently eliminated compared to symptoms previously exhibited by the cell or subject.

A viral infection in a subject may be symptomatic or asymptomatic. A symptomatic viral infection may result in clinical symptoms in a subject infected with the virus that may be detected and assessed using an embodiment of a method of the invention. Non-limiting examples of clinical symptoms include, but are not limited to, fever, shortness of breath, difficulty breathing, loss of sense of taste and/or smell, low blood oxygenation saturation, chills, vomiting, diarrhea, headache, muscle aches/pain, weakness, loss of appetite, malaise, nasal congestion, body aches, cough, sore throat, runny nose, and sneezing. It will be understood that presence, absence, and/or severity of one or more symptoms of a viral infection may be determined and/or assessed in an infected subject. Severity of a viral infection varies with different viruses and in different subjects. For example, a first subject with a viral infection may exhibit one or more symptoms such as, fever, chills, cough, etc. and a second subject with a more severe infection with the virus may exhibit some or all of the symptoms of the first subject, and also one or more of symptoms such as but not limited to trouble breathing, confusion, inability to stay awake, bluish lips or face, pain or pressure in chest, and significantly low blood oxygen saturation. It will be understood that clinical symptoms in a subject with a viral infection can be assessed using routine procedures and the symptoms identified by a health-care professional.

### Reducing Viral Titers

Some embodiments of methods of the invention include increasing a level of N-N dimers in virions in a cell or subject. It now has been determined that the presence of N-N dimers, which result from the presence of a modified N protein of the invention, can significantly reduce viral titers in the cell or subject. Although not wishing to be bound by a particular theory, it has now been identified that the presence of the N-N dimer in viral particles interferes with interaction between N protein and NSP3 protein and that mismatches between the viral N linker domain and the NSP3 Ubl1 domain severely attenuate viral titers and/or block viral rescue. Reducing viral titer in a subject can result in a less severe viral infection and can also reduce a level of contagiousness of the subject with the viral infection.

### Nucleic Acids

As used herein, the term “nucleic acid,” “nucleic acid molecule,” or “polynucleotide” refers to a polymer comprising multiple nucleotide monomers. The term “nucleotide” as used herein includes a phosphoric ester of nucleoside—the basic structural unit of nucleic acids (DNA or RNA). A nucleic acid may be either single stranded, or double stranded with each strand having a 5′ end and a 3′ end. A nucleic acid may be RNA (including but not limited to mRNA or genomic RNA of an RNA virus), DNA (including but not limited to cDNA, genomic DNA, or genomic DNA of a DNA virus), or hybrid polymers (e.g., DNA/RNA). The terms “nucleic acid” and “nucleic acid molecule” do not refer to any particular length of polymer. Nucleic acid molecules of the invention may be at least 100, 200, 300, 400, 500, 600, 700, 800, 900, 1000, 2000, 5000, or 10,000 nucleotides in length. The term “sequence,” used herein in reference to a nucleic acid molecule, refers to a contiguous series of nucleotides that are joined by covalent bonds, such as phosphodiester bonds. A nucleic acid molecule may be chemically or biochemically synthesized or may be produced by or isolated from a subject, cell, tissue, or other biological sample or source that comprises, or is believed to comprise, nucleic acid sequences including, but not limited to RNA, mRNA, and DNA. Further, this disclosure contemplates that a nucleic acid molecule of the invention may comprise at least one modified nucleotide, which may be incorporated into a polynucleotide by, for example, chemical synthesis. Such modified nucleotides may confer additional desirable properties absent or lacking in the natural nucleotides, and polynucleotides comprising modified nucleotides may be used in the compositions and methods of the invention.

It is known in the art that different organisms exhibit bias towards use of certain codons over others for the same amino acid. Therefore, in some embodiments of the invention, sequences of nucleic acid molecules of the invention are codon-optimized, meaning that the codons of the nucleic acid sequence are tailored for the codon preferences of the organism in which the nucleic acid molecule will be expressed. In some embodiments, sequences of nucleic acid molecules of the invention are human-codon-optimized, i.e., optimized for expression in human cells.

### Proteins and Fusion Proteins—Full Length, Functional Variants, and Functional Fragments

Aspects of the invention include compositions encoding and methods using full-length proteins or functional fragments thereof. The terms “protein” and “polypeptide” are used interchangeably herein and thus the term polypeptide may be used to refer to a full-length protein and may also be used to refer to a fragment of a full-length protein. A protein is a polymer of amino acids, and as used herein refers to at least two amino acids. These properties included the ability to evade prior immunity, increased transmission and altered virulence.

The term “variant” as used herein in the context of proteins and/or polynucleotide molecules, describes a molecule with one or more of the following characteristics: (1) the variant differs in sequence from the molecule of which it is a variant (also referred to herein as a “parent molecule”), (2) the variant is a fragment of the molecule of which it is a variant and is identical in sequence to the fragment of which it is a variant, and/or (3) the variant is a fragment and differs in sequence from the fragment of the molecule of which it is a variant. As used herein, the term “parent” in reference to a sequence means a sequence from which a variant originates. The term “functional” used in reference to a variant of a modified N protein of the invention, means the variant of the modified N protein variant can form an N-N dimer.

A functional variant of a modified full-length N protein or functional fragment thereof of the invention includes an R→C or G→C substitution present in the modified N protein, (non-limiting examples of which are G215C, G214C, R185C, G243C substitutions) and also includes one or more additional substitutions, deletions, point mutations, truncations, and/or additions of amino acids or non-amino acid moieties. A functional variant of a modified N protein sequence of the invention may be the modified N protein sequence or its encoding polynucleotide sequence that has an additional change of 1, 2, 3, 4, 5, 6, 7, 8, 9, 10, 11, 12, 13, 14, 15, 16, 17, 18, 19, 20, 21, 22, 23, 24, 25, or more amino acids or nucleic acids, respectively in the sequence as compared to the parent modified polypeptide, or its encoding nucleic acid sequence. As used herein, a sequence change may be one or more of a substitution, deletion, insertion, or a combination thereof. As a non-limiting example, the amino acid sequence of a functional variant of a modified N protein of the invention may be identical to the sequence set forth as SEQ ID NO: 6 except the functional variant of the modified N protein variant has one or more additional amino acid substitutions, deletions, insertions, or combinations thereof, and can form an N-N dimer.

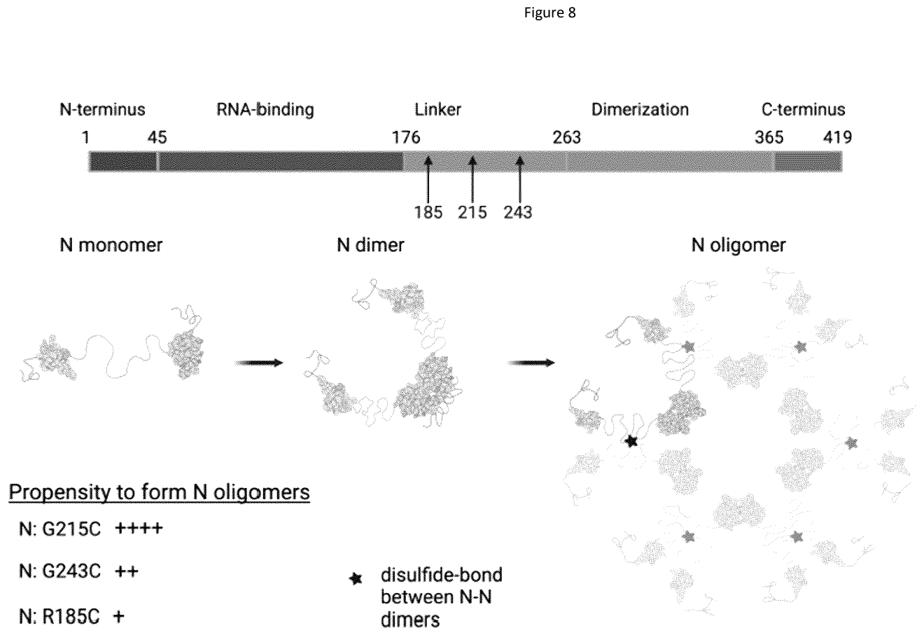

As used herein in reference to a modified N protein of the invention, the term “functional fragment” means a fragment of a modified N protein of the invention that can form a stable dimer. In some embodiments, a functional fragment comprises a linker domain and a dimerization domain of a modified N protein of the invention. FIG. 8 provides an illustration of domains of a SARS-COV-2 N protein, which include an RNA-binding domain and dimerization domains, interspersed with flexible unstructured regions at the N and C-termini and a linker region in the middle of the N protein. As non-limiting examples, fragments of modified N proteins of the invention set forth as one of SEQ ID NOs: 2-8 are provided herein as SEQ ID NOs: 30-36, respectively. Each fragment set forth as one of SEQ ID NOs: 30-36 comprises the linker region and dimerization domain of its parent sequence. Using routine methods in combination with the sequences and disclosure provided herein, a skilled artisan can prepare additional functional fragments of modified N proteins of the invention.

In some embodiments, a functional fragment comprises a modified N protein set forth herein but with 1, 2, 3, 4, 5, 6 or more amino acid additions, deletions, and/or substitutions in the N terminal region of the modified N protein. In certain embodiments, a functional fragment comprises a modified N protein set forth herein but with 1, 2, 3, 4, 5, 6 or more amino acid additions, deletions, and/or substitutions in the C terminal region of the modified N protein. In certain embodiments, a functional fragment comprises a modified N protein set forth herein but with 1, 2, 3, 4, 5, 6 or more amino acid additions, deletions, and/or substitutions in the N terminal region of the modified N protein and 1, 2, 3, 4, 5, 6, or more amino acid additions, deletions, and/or substitutions in the C terminal region of the modified N protein. FIG. 8 shows the N and C termini of a SARS-COV-2 N protein (SEQ ID NO: 1). The N terminal region or domain of SEQ ID NO: 1 includes amino acids from 1 to 45 and the C terminal region or domain of SEQ ID NO: 1 includes amino acids from 365 through 419. The linker region of SEQ ID NO: 1 includes amino acids from 176 to 263. The dimerization domain of SEQ ID NO: 1 includes amino acids from 263 to 365. It will be understood that amino acids of N and C termini, RNA binding region, linker region, and dimerization regions of other modified N proteins of the invention can be determined using routine sequence alignment methods.

A “functional fragment” of a modified N proteins of the invention is a fragment of a full-length modified N protein that retains at least a portion of a distinct functional capability of the polypeptide. A portion is at least 5%, 10%, 15%, 20%, 25%, 30%, 35%, 40%, 45%, 50%, 55%, 60%, 65%, 70%, 75%, 80%, 85%, 90%, 95%, or 100% of the functional capability (including all values within the range). Functional capabilities that can be retained in a fragment include forming a dimer with a full-length modified N protein or a fragment of a full-length modified N protein. In some embodiments, a functional fragment of a polypeptide may be encoded by a nucleic acid molecule of the invention, or may be synthesized using art-known methods, and tested for function using the methods exemplified herein. Full-length proteins and functional fragments thereof that are useful in methods and compositions of the invention may be recombinant polypeptides.

A fragment of a full-length polypeptide may comprise at least up to n-1 contiguous amino acids of the full-length polypeptide having a consecutive sequence found in a wild-type polypeptide or in a modified polypeptide sequence as described herein (with “n” equal to the number of amino acids in the full-length polypeptide). Thus, for example, a fragment of a 419 amino acid-long polypeptide would be at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 1632, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 230, 240, 250, 2.60, 270, 280, 290, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, or 418 contiguous amino acids of the 419 amino acid polypeptide. The term “functional” used in reference to a fragment of a modified N protein of the invention, means the fragment of the modified N protein can (a) form an N-N dimer with another of the same functional fragment of the modified N protein; (b) form an N-N dimer with another functional fragment of the modified N protein; and/or (c) form an N-N dimer with a full-length modified N protein.

Identity of related proteins, such as a parent protein and a variant thereof and/or a parent protein and a functional fragment thereof, can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide of the invention has at least 70%, 75%, 80%, 85%, 90%, 91%, 92%, 93%, 94%, 95%, 96%, 97%, 98%, 99% but less than 100% sequence identity to the aligned region of its “parent” reference polynucleotide or polypeptide as determined by sequence alignment programs and parameters described herein and known to those skilled in the art. Such tools for alignment include those of the BLAST suite (Stephen F. Altschul, et al. (1997).

It will be understood that with respect to variants of a modified N protein, certain regions of an N protein may be more tolerant of sequence modification than other regions of the modified N protein. For example, a variant of a modified N protein of the invention may have additional sequence modifications in the N terminal region and/or C terminal region of its parent modified N protein and modifications in those regions may be better tolerated than modifications made in other regions of that modified N protein. In some embodiments, the amino acid sequence of the N terminal region of a variant of a modified N protein of the invention has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the N terminal region of its parent modified N protein sequence. In some embodiments, the amino acid sequence of the C terminal region of a variant of a modified N protein of the invention has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the C terminal region of its parent modified N protein sequence.

Regions of a modified N protein that may be less tolerant of additional modifications than the N and C termini are the linking domain and the dimerization domain. In some embodiments, the amino acid sequence of the linking domain of a variant of a modified N protein of the invention has at least 85%, 86%, 87%, 88%, 89%, 90%, 91%, 92%, 93%, 94%, 95%, 96%, 97%, 98%, 99%, or 100% sequence identity to the linking domain of its parent modified N protein sequence. In some embodiments, the amino acid sequence of the dimerization domain of a variant of a modified N protein of the invention has at least 85%, 86%, 87%, 88%, 89%, 90%, 91%, 92%, 93%, 94%, 95%, 96%, 97%, 98%, 99%, or 100% sequence identity to the dimerization domain of its parent modified N protein sequence. It will be understood that variants of a modified N protein sequence of the invention may have additional modifications in one, two, three, four, or more regions of the modified N protein sequence. As a non-limiting example, a functional variant of a modified N protein sequence of the invention may have 1, 2, 3, 4 or more substitutions, deletions, and/or additions in one or more of the N terminal region, the linking domain, the RNA binding domain, the dimerization domain, and the C terminal region.

The skilled artisan will realize that conservative amino acid substitutions may be made in a modified N protein of the invention to provide a functionally equivalent modified N protein that retains a functional capability of a modified N protein that does not have the conservative amino acid substitution(s) in the functionally equivalent modified N protein. As used herein, a “conservative amino acid substitution” refers to an amino acid substitution that does not alter the relative charge or size characteristics of the polypeptide in which the amino acid substitution is made. Modified polypeptides can be prepared according to methods for altering polypeptide sequence and known to one of ordinary skill in the art such. Exemplary functionally equivalent polypeptides include conservative amino acid substitutions of an N protein or functional fragment thereof. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. One of ordinary skill in the art will know how to use 1, 2, 3, 4, 5 or more conservative amino acid substitutions in conjunction with methods and compositions of the invention.

In certain aspects, compositions and methods of the invention include a fusion protein encoded by a nucleic acid molecule of the invention. As used herein, “fusion protein” refers to a non-naturally occurring protein comprising amino acid sequences from at least two different proteins. In some embodiments, the fusion protein includes a modified N protein of the invention or a functional fragment of the modified N protein, or a functional variant thereof that can form an N-N dimer, either with a full-length modified N protein or functional variant thereof or with a functional fragment of the modified N protein of the invention or a functional variant thereof. In certain embodiments of the invention, the fusion protein comprises any one of SEQ ID NO: 2, 3, 4, 5, 6, 7, 8, or a functional fragment of any one of SEQ ID NO: 2, 3, 4, 5, 6, 7, and 8.

### Viral Immunization Preparations

Methods and compositions of the invention may be used as viral immunization preparations, which are also referred to herein as anti-virus vaccines, or vaccines. Certain embodiments of invention include vaccines against a virus. Examples of viral immunization preparations of the invention include but are not limited to a viral immunization preparation comprising a modified N protein of the invention or a functional fragment thereof, an mRNA vaccine, a DNA vaccine, and a vector viral vaccine. In some embodiments, a viral immunization preparation of the invention comprises a live attenuated virus comprising a modified N protein of the invention or functional fragment thereof, or an inactivated (killed) virus comprising a modified N protein of the invention or functional fragment thereof. In some embodiments, a vaccine is an RNA (e.g., mRNA) vaccine. Non-limiting examples of other vaccines of the invention are live/attenuated viral vaccines, killed/inactivated viral vaccines, subunit vaccines, protein antigen vaccines, DNA vaccines, VLP vaccines, etc.

A viral immunization preparation of the invention may be produced using art-known procedures in conjunction with information disclosed herein about modified N proteins of the invention and their encoding polynucleotides, functional variants of modified N proteins of the invention and their encoding polynucleotides, and functional fragments of modified N proteins of the invention and their encoding polynucleotides. Routine methods for preparing a viral immunization preparation are set forth, for example in: Hill, A. et al., Methods Mol Biol 2021:2183:1-8; Zimmerman, P. & Curtis, N. Clin Microbiol Rev. 2019 Mar. 13; 32 (2): e00084-18; and Pulendran, B. & Ahmed, R. Nat Immunol. 2011 June; 12 (6): 509-17. The content of each of which is incorporated herein by reference in its entirety.

In some embodiments, a composition of the invention included in a viral immunization preparation is a modified viral N protein or functional fragment thereof. In some embodiments of the invention a modified viral N protein is a modified coronavirus N protein. In some embodiments, a modified viral N protein is a modified betacoronavirus N protein. In certain embodiments of the invention, a modified viral N protein is a modified SARs N protein or a modified MERs N protein. Non-limiting examples of modified viral N proteins are set forth herein as SEQ ID NOs: 2, 3, 4, 5, 6, 7, and 8. In some embodiments, a composition of the invention included in a viral immunization preparation is a functional fragment of a modified viral N protein. Use part of the virus. Methods of producing a viral immunization preparation are known and routinely practiced in the art. In some embodiments, a viral immunization preparation of the invention comprises a modified N protein of the invention or a functional fragment thereof. With this approach, part of the virus, the modified N protein or a functional fragment is used as a vaccine.

In some embodiments, a viral immunization preparation of the invention is an mRNA vaccine comprising mRNA that when transcribed produces a modified N protein of the invention, or a functional fragment thereof. In certain embodiments, a viral immunization preparation of the invention is a DNA vaccine, comprising the genetic code from which the mRNA that when transcribed produces a modified N protein of the invention, or a functional fragment thereof. In some embodiments, a viral immunization preparation of the invention comprises a vector virus vaccine. In some embodiments, a viral immunization preparation of the invention comprises a whole-inactivated virus, in which intact virions are used as the antigen, after they have been inactivated (i.e., with heat, irradiation or chemical fixation).

### Methods of Treatment

Provided herein are compositions (e.g., pharmaceutical compositions), methods, kits, and reagents for prevention and/or treatment of viral infections in humans and other mammals. A vaccine of the invention can be used as therapeutic agent, which, as used herein means an agent that can prevent and/or reduce an infection by a virus. As a non-limiting example, an RNA (e.g., mRNA) vaccine or a protein vaccine of the invention, can be used as a therapeutic agent against infection by a virus. It will be understood that a viral immunization preparation of the invention can be used to immunize a subject against the virus the immunization preparation was produced to target. As a non-limiting example, a viral immunization preparation of the invention comprising a modified N protein of a SARs-COV-2 virus, may be used as a therapeutic agent against infection by the SARs-COV-2 virus. As another non-limiting example, a viral immunization preparation of the invention comprising a modified N protein of a MERs virus, is administered to a subject as a therapeutic agent against infection in the subject by the MERS virus. Prevention and/or reduction of a viral infection can result from administering viral immunization preparation of the invention to a subject. A viral immunization preparation of the invention (also referred to herein as a vaccine of the invention) can be administered to a subject, 1, 2, 3, 4, 5, 6, 7, 8, 9, 10, or more times. In some embodiments, a vaccine of the invention is administered to a subject believed to be at risk of exposure to the virus against which the vaccine has been produced and in certain embodiments a vaccine of the invention is administered to a subject known to have been exposed to the virus against which the vaccine has been produced. In certain embodiments, a vaccine of the invention is administered to a subject known to be infected with the virus against which the vaccine was produced. In some embodiments of methods of the invention, a viral immunization preparation of the invention is administered to a subject to prevent and/or treat infection in the subject by a virus that is not the virus against which the vaccine was produced. In some embodiments the virus that is not the virus against which the vaccine was produced is a variant of the virus against which the vaccine was produced with sufficient similarity for the vaccine to have efficacy against the variant virus.

A method of eliciting an immune response in a subject against a virus is provided in certain embodiments of the invention. In some embodiments, a method of the invention comprises administering to a subject viral immunization preparation of the invention comprising a modified viral N protein of the invention or functional fragment thereof, or a polynucleotide encoding a modified N protein of the invention or a functional fragment thereof, thereby inducing in the subject an immune response specific to the virus against which the viral immunization preparation was produced. In some embodiments, following administration of a viral immunization preparation of the invention to a subject, an anti-antigenic protein antibody titer in the subject is increased as compared to a control. In some embodiments a control is an anti-antigenic protein antibody titer in a subject administered a different vaccine against the virus. In certain embodiments, a control is an anti-antigenic protein antibody titer in a subject to whom the vaccine against the virus is not administered. As used herein, the term “anti-antigenic protein antibody” means a serum antibody the binds specifically to the antigenic protein.

In some embodiments, vaccine of the invention capable of eliciting an immune response is administered to a subject intramuscularly, subcutaneously, orally or as a nasal spray.

An effective amount of a vaccine of the invention is an amount that prevents and/or reduces the viral infection in the subject to whom the vaccine is administered. In some embodiments of methods of the invention, administering an effective amount of a vaccine of the invention increases an anti-antigenic protein antibody titer in the subject between 1 log to 10 log following vaccination compared to control anti-antigenic protein antibody titer in a subject, which may be a level of anti-antigenic protein antibody titer in a subject vaccinated with a different vaccine against the virus or a level of anti-antigenic protein antibody titer in a subject not vaccinated against the virus.

In some embodiments the anti-antigenic protein antibody titer in a subject administered a vaccine of the invention is increased 1 log, 2 log, 3 log, 5 log, 6 log, 7 log, 8 log, 9 log, or 10 log following vaccination compared to a control anti-antigenic protein antibody titer in a subject, which may be a level of anti-antigenic protein antibody titer in a subject vaccinated with a different vaccine against the virus or a level of anti-antigenic protein antibody titer in a subject not vaccinated against the virus. In some embodiments of the invention, a level of an immune response in a subject may be assessed using routine methods, for example by determining a level of anti-antigenic protein antibody titer in the subject.

### Subjects and Cells

As used herein, a subject shall mean a vertebrate animal including but not limited to a human, mouse, rat, guinea pig, rabbit, cow, dog, cat, horse, goat, and non-human primate, e.g., monkey. In certain aspects of the invention, a subject may be a domesticated animal, a wild animal, or an agricultural animal. Thus, the invention can be used to treat diseases or conditions in human and non-human subjects. For instance, methods and compositions of the invention can be used in veterinary applications as well as in human prevention and treatment regimens. In some embodiments of the invention, the subject is a human. A subject may be considered to be a normal subject or may be a subject known to have or suspected of having a viral infection. Non-limiting examples of viral infections are SARS-COV, SARS-COV-2, SARS-COV-1, MERS, etc.

Some aspects of the invention include cells used in conjunction with compositions and methods of the invention. For example, though not intended to be limiting, a culture of cells may be contacted with a viral immunization preparation of the invention to assess an effect of the viral immunization preparation on the cells. Non-limiting examples of cells that may be used in methods and/or to test compositions and/or preparations of the invention are human embryonic kidney (HEK) cells, a non-limiting example of which are HEK-293T/17 cells; and Vero-E6 Cells. Certain embodiments of the invention include use of mammalian cells; including but not limited to cells of humans, non-human primates, dogs, cats, horses, rodents, etc. In some embodiments of the invention, cells that are used are non-mammalian cells; including but not limited to insect cells, avian cells, fish cells, plant cells, etc.

In some embodiments, a cell used in conjunction with a method, composition, and/or preparation of the invention is a healthy normal cell that is not known to have or suspected of having a viral infection. In some embodiments, a cell used in conjunction with a method, composition, and/or preparation of the invention is a cell that is infected with a virus. Non limiting examples of elements of an abnormal cell are: (1) a cell that has a viral infection; (2) a cell obtained from a subject that has, had, or is suspected of having a viral infection; and (3) a cell that is a model for a viral infection, etc. In some embodiments of the invention, a cell is a control cell.

### Controls

Certain embodiments of compositions and methods of the invention used to prepare a viral immunization preparation of the invention to suppress a viral infection. Some embodiments of the invention comprise comparing a therapeutic result obtained for a subject, with a control value obtained from one or a plurality of control subjects. As used herein the term “plurality” means two or more. As a non-limiting example, some embodiments of the invention include contacting a plurality of cells with either a composition of the invention comprising a modified N protein or with a composition that does not contain the modified N protein, infecting all cells with SARS-COV-2, and determining the respective proportions of infected cells. A control may also be a no-treatment control, wherein a cell or subject does not receive a treatment. As another non-limiting example, some embodiments of the invention include administering to a subject a composition comprising a viral immunization preparation of the invention that includes modified N protein, and comparing infection of the subject with the virus against which the viral immunization preparation was produced with the viral infection in a subject or plurality of subjects who have not been administered the composition comprising the viral immunization preparation of the invention.

As used herein a control may be as described above and, in addition, may be a predetermined value, which can take a variety of forms. It can be a single cut-off value, such as a median or mean, or a reference value or range of values. In some embodiments of the invention, a control value is a value determined previously for a subject during the course of infection and/or treatment of the subject. A control can be established based upon comparative groups such as a cell (in vitro or in a subject) that is contacted with a viral immunization preparation of the invention, compared to a cell or subject that under substantially identical conditions is not contacted with, or is contacted with a different amount of the viral immunization preparation of the invention. Another example of comparative groups may include a cell or subject that has a disorder or condition and a cell or subject without the disorder or condition. Another comparative group may be a subject or cells from a subject with a family history of a disease or condition and a subject or cells from a subject without such a family history. Other examples of comparative groups may include, but are not limited to cells or subjects that have a severe viral infection; cells or subjects that do not have a severe viral infection; cells or subjects that are asymptomatic for a viral infection, etc. Those in the art will readily identify suitable control cells and subjects for use with compositions and methods of the invention. In some embodiments, a control may be a placebo, an inactive substance used to compare results with a composition or method of the invention.

### Examples

### Example 1. Assays for In Vitro Function

### Materials & Methods

### Ethics Statement

Research conducted in this study was reviewed and approved by the Institutional Biosafety Committee of the University of Vermont (REG202100001) and the Institutional Biosafety Committee of the University of Texas Medical Branch (UTMB). All studies in animals were conducted under a protocol approved by the UTMB Institutional Animal Care and Use Committee and complied with USDA guidelines in a laboratory accredited by the Association for Assessment and Accreditation of Laboratory Animal Care. UTMB is a registered Research Facility under the Animal Welfare Act. It has a current assurance (A3314-01) with the Office of Laboratory Animal Welfare (OLAW), in compliance with NIH Policy. Procedures involving infectious SARS-COV-2 were performed in the Galveston National Laboratory ABSL3 facility.

### Cell Culture

### Stable Cell Lines

Human embryonic kidney cells (HEK-293T/17) (CRL-11268, American Type culture Collection, Manassas, VA) were kindly provided by J. Salogiannis (UVM); Vero-E6 cells expressing TMPRSS2 were obtained from the Japanese Cancer Research Resources Bank (JCRB1819). All cell lines were maintained in Dulbecco's Modified Eagle Medium (DMEM) (10-017-CM, Corning) and supplemented with 10% fetal bovine serum (FBS) (16140-071, Gibco). Cells were grown at 37° C. and 5% CO₂.

### Primary Cells

24-well transwell inserts (3470-Clear, Costar) were coated with 125 μL of a 50 ug/mL rat tail collagen (A10483-01, Gibco) in 0.02N acetic acid for 1 hr at room temperature. Collagen solution was aspirated and washed once with DMEM (10-017-CM, Corning) before membranes were equilibrated with DMEM in both the apical and basolateral chambers for 1 hr at 37° C. DMEM was replaced with “basal media” containing PenumaCult Ex-Plus medium (05040, Stemcell Technologies), 10 mL of PneumaCult 50× supplement (05042, StemCell Technologies), 0.001% hydrocortisone (07926, StemCell Technologies), 1% penicillin/streptomycin (30-001-Cl, Corning), 30 ug/mL gentamicin (15750060, Gibco) and 15 ug/mL amphotericin (30-003—CF, Corning) for 20 mins at 37° C. Expanded human bronchial epithelia cells (CC-2541, Lonza) were plated onto transwell inserts at a density of 40,000 cells/cm2 in warmed basal media. Apical media was changed the next day to remove residual DMSO after which apical and basolateral media was changed every other day until cells were 98-100% confluent. Once at confluency, basal media was aspirated from the apical side and the basolateral chamber media was replaced with PneumaCult-ALI S media (05002, StemCell Technologies), 0.002% heparin (07980, StemCell Technologies), 0.001% hydrocortisone, 1% penicillin/streptomycin, 30 ug/mL gentamicin, 10% v/v PneumaCult-ALI supplement (05003, StemCell Technologies), 1% v/v PneumaCult-ALI maintenance Supplement (05006 StemCell Technologies), and 15 ug/mL amphotericin. Basolateral chamber media was changed every other day until differentiation occurred, approximately 21 days post airlift.

### Viruses

### Viral Isolation

SARS-COV-2 WA1/2020 was obtained from the World Reference Center for Emerging Viruses and Arboviruses at the University of Texas Medical Branch. Viral isolates for Alpha, Beta, Gamma, Delta, Epsilon, Iota, Mu, and Omicron SARS-COV-2 variants were obtained from primary clinical specimens. Nasal swabs were acquired from patients who tested positive for COVID-19 and the variant lineage was determined via Next Generation Sequencing (NGS) or digital droplet PCR either before or after isolation [Johnson, B. A. et al., PLOS Pathog. 18, e1010627 (2022)]. All isolated specimens were less than eight days old before −80° C. storage, stored in saline solution, and had a diagnostic polymerase-chain reaction (PCR) cycle threshold of less than 32. The use of deidentified positive specimens was declared exempt by the University of Vermont Institutional Review Board and the University of Washington Institutional Review Board (STUDY00010205). Virus from clinical specimens was isolated in VeroE6-TMPRSS2 cells. Cells were monitored daily for cytopathic effect (CPE) and harvested when ˜50% of the cells exhibited CPE or death. Clarified samples were stored at −80° C. and used to generate working viral stocks. Viral stocks were titered by focus forming assay. Next generation sequencing of viral stocks was conducted by the Microbial Genome Sequencing Center or SeqCoast Genomics.

### Construction of Recombinant SARS-COV-2

The sequence of recombinant wild-type (WT) SARS-COV-2 is based on the USA-WA1/2020 strain provided by the World Reference Center for Emerging Viruses and Arboviruses (WRCEVA) and originally isolated by the USA Centers for Disease Control and Prevention [Harcourt, J. et al. Severe Acute Respiratory Syndrome Coronavirus 2 from Patient with Coronavirus Disease, United States—Volume 26, Number 6—June 2020—Emerging Infectious Diseases journal-CDC. doi: 10.3201/eid2606.200516]. Recombinant WT SARS-CoV-2 and mutant viruses were created using a cDNA clone and standard cloning techniques as described previously [Xie, X. et al. Cell Host Microbe 27, 841-848.e3 (2020) and Xie, X. et al. Nat. Protoc. 16, 1761-1784 (2021)]. Construction of WT SARS-COV-2 and mutant viruses were approved by the University of Texas Medical Branch Biosafety Committee.

### In Vitro SARS-COV-2 Infection

### Vero-TMPRSS2 Cells

Vero-TMPRSS2 cells were seeded in a 24 well plate at 50,000 cells/well one day before infection. The following day, cells were inoculated in 100 μl of virus at the indicated MOI for one hour at 37° C. and 5% CO₂, after which the inoculum was removed and replaced with fresh growth media. Supernatants were collected at the indicated timepoints, clarified to remove cellular debris, and stored at −80° C. until time of titering via focus assay.

### Human Bronchial Epithelial Cells (HBECs)

Differentiated HBECs cells were washed three times with ˜200 μl of 37C HEPES buffered saline before being infected with 100 μl of virus at an MOI of 0.5 resuspended in ExPneumaCult ALI-S media+supplements (see primary cell maintenance section). After a 1 hr incubation at 37° C. and 5% CO₂, supernatant was removed. The apical side of the cells were washed with 150 μl ExPneumaCult ALI-S media+supplements at the indicated times post infection to collect virus. Viral washes were stored at −80° C. until time of titering via focus assay. Basal media was changed every 48 hrs during the infection time course.

### Focus Forming Assay

Viral titrations were performed largely as previously described [Despres, H. W. et al. Proc. Natl. Acad. Sci. 119, e2116518119 (2022)]. VeroE6-TMPRSS2 cells were seeded at a density of 60,000 cells/well into white 96-well plates (Falcon; #353296). 24 hrs later, viral samples of interest were serially diluted 10-fold in DMEM+10% FBS. Plates were aspirated and infected with diluted viral samples for 1 hr at 37° C. and 5% CO₂ before being overlaid with a 1.2% methylcellulose (Acros; #332620010) solution suspended in DMEM. After 24 hrs, the methylcellulose solution was aspirated, and the plates were fixed a 4% formaldehyde solution (Honeywell; #F1635-4L) for 20 mins before being washed once with deionized (DI) water. Cells were permeabilized in 50 μl of 0.05% Triton X100 (Fisher; #BP151-100) for 5 mins, washed with PBS and blocked (5% non-fat milk solution in PBS) for 1 hr at room temperature. Virally infected cells were detected with an anti-SARS-COV-2 N antibody (Sinobiological; #40143-R001, 1:20,000) resuspended in 5% milk for 1 hr at 37° C. Cells were washed twice with PBS and stained with an HRP-conjugated secondary antibody (Seracare; #5220-0337, 1:4,000) resuspended in 5% milk for 1 hr at 37° C. Cells were washed twice with PBS, and foci developed using a TruBlue HRP substrate (SeraCare; #5510-0030). Foci were imaged on a BioTek ImmunoSpot S6 MACRO Analyzer and manually counted.

### Viral Concentration

Viral stocks were concentrated by vortexing 1 ml of high titer viral stocks with 10% polyethylene glycol (PEG) (Sigma-Aldrich; #P6667) for ˜5-10 minutes before centrifugation at 10,000 g for 30 mins at 4° C. Pellets were then resuspended in a NP-40, 1% Trition X-100 lysis buffer (see SDS-PAGE and western blotting section below).

### Analysis of Publicly Available Genomic Data

The identity of the amino acid residue in the nucleocapsid protein (including at 215) in 7,342,041 SARS-COV-2 sequences deposited in GenBank, the China National Center for Bioinformation and from COG-UK were analyzed using the phylogenetic tree (Cov2Tree) and visualized by Taxonium [Sanderson, T. eLife 11, e82392 (2022) and Kramer, A. M., et al., Bioinformatics 39, btac772 (2023)]. Sequences for non-SARS-COV-2 coronavirus nucleocapsid sequences were obtained through the NIH Protein Databank and aligned using Clustal Omega (EMBL-EBI, Clustal 0 (1.2.4)) [Madeira, F. et al. Nucleic Acids Res. 50, W276-W279 (2022)].

### Hamster Infection Studies

For in vivo studies, three- to four-week-old male hamsters were purchased from Envigio, and all studies conducted within the Galveston National Laboratory ABSL3 facility. Studies were conducted in accordance with a protocol approved by the University of Texas Medical Branch Institutional Animal Care and Use Committee and comply with the United States Department of Agriculture guidelines. All laboratories were accredited by the Association for Assessment and Accreditation of Laboratory Animal Care. For infection studies, animals housed in groups of five were intranasally infected PBS alone (mock) or with 10⁴ FFU of WT or N: G215C mutant viruses. Animals were then monitored daily for weight loss and development of clinical disease until completion of the experiment. All procedures were carried out under anesthesia with isoflurane (Henry Schein Animal Health), with the exception of weighing.

### Histology

Hematoxylin and eosin-stained microscopic slides of lung from each hamster were separated into three groups according to the number of days after intranasal inoculation of SARS Cov 2. Microscopic slides of each group were scrambled thoroughly and examined blindly to assess the severity of pathologic lesions. Slides were placed in the order of serial pairwise comparison of the extent of lesions. Upon completion of the ordering of severity, numbers were assigned to the slides from 1, least pathologic change, to the highest number for the slide with the most severe pathology. At this point, the code was revealed, and the sum of the rank order of severity numbers for each group: mock, wild type, and mutant was calculated and divided according to the number of slides in the group to determine a severity score for the group. The number of slides in the groups varied slightly as tissues which contained abundant polymorphonuclear leukocytes, very likely indicating superimposed bacterial bronchopneumonia were removed and not scored. Separate sums of rank order scores and average severity score were calculated for each day.

### Plasmid Generation

The sequences for the N protein of our Beta, Delta, and Iota SARS-COV-2 variants, as well as the WA1/2020 (‘WT’) sequences were synthesized and subcloned into a pcDNA3.1 vector (GenScript). In addition, point mutants were generated in which the cysteine in the Beta, Iota, and Delta N sequences was mutated back to the corresponding residue in WA1 (Beta: C185R, Delta: C215G, Iota: C243G) or where cysteine was introduced at the 185, 215 or 243 positions into the WA1 background (R185C, G215C, G243C). Plasmid sequences were confirmed via whole-plasmid sequencing (Plasmidsaurus).

### Transfection

HEK-293T cells were seeded at a density of 350,000 cells/well in a 12-well plate (Corning #3512). The following day, 500 ng of the appropriate plasmid and 2 μl of Lipofectamine (Invitrogen #52887) diluted to a volume of 40 μl in DMEM were incubated for 24 hrs at 37° C. and 5% CO₂. Cells were harvested by scraping into PBS, pelleted, and lysed for SDS-PAGE and western blotting (see additional information below).

### SDS-PAGE and Western Blotting

Cells were lysed in a NP-40 lysis buffer (Thermo Scientific #J60766.AK) containing a 1% Triton X-100 solution (Fisher, #BP151-100) and protease inhibitors (Thermo Scientific Fisher #A32955) for 20 mins on ice. Cell lysates were clarified by spinning at 14,000 rpm for 10 mins at 4° C. to remove insoluble debris and diluted in a 1:1 ratio (v/v) of 4X Laemmli sample buffer (250 mM Tris-HCl pH 6.8,40% glycerol, 8% SDS, 0.04% Bromophenol Blue). Reduced lysates were treated with either 2.75 mM 2-mercaptoethanol (Gibco #21985-023) or 10 mM dithiothreitol (Fisher Bioreagents #BP172-5). For NEM processing, samples were lysed in 1.25 M n-ethylmaleimide (NEM; Thermo Fisher #23030 and incubated at 4° C. overnight. All samples were boiled for 5 mins before loading. Samples were separated in NuPAGE 4-12% Bis-Tris gels (Invitrogen #NPO335BOX) in MES buffer (Invitrogen #NP0002) with a molecular mass ladder (Thermo Fisher #LC5925) at 180v for 50 mins before being transferred into a nitrocellulose membrane (Invitrogen #IB23001) using an iBlot 2 machine (Invitrogen) at 20V for 7 mins.

Membranes were blocked in a 5% milk/PBS solution for 30 mins and then incubated overnight at 4° C. in a solution containing the primary antibody (see Table 1) in a 5% milk/PBST (0.2% Tween 20 (Thermo Fisher Scientific, #J20605-AP). Membranes were washed in PBST, incubated while rocking with secondary antibodies diluted in 5% milk/PBST for 45 min, washed again with PBST and imaged with a LI-COR Odyssey CLx. Protein expression was analyzed by measuring band densitometry in Fiji (22743772).

TABLE 1
Antibodies

| | | |
|---|---|---|
| | | Dilution |
| Species, Target | Catalog #, Company | Used |

Primary Antibodies

| | | |
|---|---|---|
| SARS-CoV-2 nucleocapsid | Invitrogen, MA5-35943 | 1:1,000 |
| protein (mouse) | | |
| SARS-CoV-2 nucleocapsid | SinoBiological, | 1:10,000 |
| protein (rabbit) | 40143-R001 | (western) |
| | | 1:20,000 (focus |
| | | assay) |
| SARS-CoV-2 membrane (M) | Cell Signaling, E5A8A | 1:1000 |
| protein (mouse) | | |
| β-actin (mouse) | Novus Biologicals, | 1:10,000 |
| | NB600-501 | |

Secondary Antibodies

| | | |
|---|---|---|
| IRDye 680LT goat anti-rabbit | LICOR, #92668021 | 1:10,000 |
| IgG | | |
| IRDye 680RD goat anti- | LICOR, #92668070 | 1:10,000 |
| mouse IgG | | |
| IRDye 800CW goat anti- | LICOR, #92632210 | 1:10,000 |
| mouse IgG | | |
| HRP anti-rabbit | Seracare; #5220-0337 | 1:4,000 |

Immunoprecipitation Assays

VeroE6-TMPRSS2 cells were infected at an MOI of 0.01 with WT (WA1) or Delta SARS-COV-2.24 hpi cells were scraped into PBS, pelleted, and lysed in NP-40/Triton lysis buffer (see SDS-PAGE and western blotting. SARS-COV-2 N was affinity purified using 10 μl of an anti-SARS-COV-2 N antibody (40143-R001, Sinobiological) and Protein G-conjugated Dynabeads (Invitrogen, #10003D) according to the manufacturer instructions.

### Mass Spectrometry

After immunoprecipitation, samples were run on an 8% BOLT gel (Invitrogen, #NW00085BOX), bands were visualized via Coomassie stain (40% methanol, 20% acetic acid, 0.01% Brilliant Blue) and de-stained (in-house). Bands of interest (monomer, dimer, or full lane) were cut out of the gel, and further cut into ˜1 mm cubes before being placed into tubes for processing using Fisher brand chemicals. Gel slices were incubated in HPLC-grade water for 5 min, and de-stained for 30 min at 37° C. (50 mM ammonium bicarbonate, 50% acetonitrile). Slices were rinsed in fresh acetonitrile for two 5 min incubations before incubating for 30 min at 55° C. in 25 mM DTT (Thermo Fisher, #R0861)/ammonium biocarbonate. Slices were cooled, incubated in 100% acetonitrile for 5 mins and incubated for 45 minutes at room temperature in 10 mM iodoacetamide (Sigma-Aldrich, #16125)/50 mM ammonium bicarbonate, in the dark. Slices were destained for 5 min, rinsed in purified water for 10 minutes and centrifuged at 13,000 rpm for 30 seconds. Next, samples were dehydrated in acetonitrile before all liquid removed and gel slices allowed to dry completely. Once dry, samples were placed on ice for 5 mins before incubating for 30 minutes on ice with trypsin (Promega #V511A)/50 mM ammonium bicarbonate and then digesting overnight at 37° C. Supernatants were removed and saved, while gel slices were covered with a 50% acetonitrile, 2.5% formic acid solution and spun for ten minutes after which supernatants were combined with those from the previous step. Finally, 30 μl acetonitrile was added to gel slices for a final 10-minute incubation, after which these supernatants were combined with those from the previous two steps and dried in a vacuum centrifuge at 37° C.

Following peptide preparation, samples were resuspended in 2.5% acetonitrile and 0.1% formic acid. Then loaded onto an EASY n-LC 1200 for HPLC (300 nL/min) followed by tandem MS/MS on an Exploris mass spectrometer (ThermoFisher). The Exploris was fitted with a Nanospray Flex ion source and chromatograph column packed in-house (35 cm×100 μm, 1.8 μm 120 A UChrom, C18 packing material). Peptides were eluted into the mass spectrometer on a 5-95% gradient of 80% acetonitrile and 0.1% formic acid over 170 minutes, followed by a 10-minute clear at 5% gradient. The following were the mass spectrometer parameters. Precursor scan range=350-1400 m/z, resolution=60,000, normalized AGC target=250%, maximum IT =25 ms. Data-dependent MS2 orbitrap resolution=15,000, normalized AGC target=50%, isolation window=±1.4 m/z, HCD collision energies=30%, dynamic exclusion=45 s with repeat count of 1. Followed by ten collision induced dissociation tandem mass spectra of the top ten most abundance ions in the precursor scan.

Resulting raw mass spectra for each sample was searched against both a forward and reverse African Green Monkey protein database and a SARS COV-2 protein database using SEQUEST [Eng J K, et al., J Am Soc Mass Spectrom. 1994; 5 (11): 976-989. doi: 10.1016/1044-0305 (94) 80016-2]. Search parameters included tryptic peptides and differential modifications (phosphorylation on serine, threonine, and tyrosine (+79.9663 Da), oxidation of methionine (+15.99.49 Da), acrylamindation of cysteine (+71.0371 Da), carboxyamidomethylation of cysteine (+57.0214 Da)). The resulting peptides lists were filtered by mass accuracy (ppm±5), cross correlation scores (z=1 XCorr≥1.8, z=2 XCorr≥2, z-3 XCorr≥2.2, z=4 XCorr≥2.4, z=5 XCorr≥2.6), and a unique delta-correlation (uniqΔcorr≥0.15). All filtering resulted in a false discovery rate of 0%. The filtered peptide lists were then compared using R software.

### Sample Preparation for Electron Microscopy

Vero-E6 cells were cultured on plates and infected with mNG WT or G215C SARS-COV-2 under BSL-3 conditions. Cells were pre-fixed with 3% glutaraldehyde, 1% paraformaldehyde, 5% sucrose in 0.1 M sodium cacodylate, then removed from the plates and transferred to Eppendorf tubes and gently pelleted. The fixative supernatant was removed, and pellets rinsed with fresh cacodylate buffer containing 10% Ficoll, placed into brass planchettes (Ted Pella, Inc.), and rapidly frozen with an HPM-010 high-pressure freezing machine (Bal-Tec, Leichtenstein). The frozen samples were transferred under liquid nitrogen to cryotubes (Nunc) containing a frozen solution of 2.5% osmium tetroxide, 0.05% uranyl acetate in acetone. Tubes were loaded into an AFS-2 freeze-substitution machine (Leica Microsystems, Vienna) and processed at −90° C. for 72 h, warmed over 12 h to −20° C., held at that temperature for 6 h, then warmed to 4° C. for 1 h. Samples were rinsed 3× with cold acetone, after which they were infiltrated with Epon-Araldite resin (Electron Microscopy Sciences) over 48 h. Cell pellets were flat-embedded between two Teflon-coated glass microscope slides and the resin was polymerized at 60° C. for 48 h.

### Electron Microscopy and Dual-Axis Tomography

Embedded cells were observed by light microscopy and appropriate blocks were extracted with a microsurgical scalpel and glued to the tips of plastic sectioning stubs. Semi-thin (170 nm) serial sections were cut with a UC6 ultramicrotome (Leica Microsystems) using a diamond knife (Diatome Ltd., Switzerland). Sections were placed on formvar-coated copper-rhodium slot grids (Electron Microscopy Sciences) and stained with 3% uranyl acetate and lead citrate. Gold beads (10 nm) were placed on both surfaces of the grid to serve as fiducial markers for subsequent image alignment. Grids were placed in a dual-axis tomography holder (Model 2040, E.A. Fischione Instruments) and imaged with a Tecnai T12-G2 transmission electron microscope operating at 120 KeV (ThermoFisher Scientific) equipped with a 2k×2k CCD camera (XP1000; Gatan, Inc.). Tomographic tilt-series and large-area montaged overviews were acquired automatically using the SerialEM software package [Mastronarde, D. N. J. Struct. Biol. 152, 36-51 (2005)]. For tomography, samples were tilted+62° and images collected at 1° intervals. The grid was then rotated 90° and a similar series taken about the orthogonal axis. Tomographic data was calculated, analyzed, and modeled using the IMOD software package [Kremer, J. R., et al., J. Struct. Biol. 116, 71-76 (1996); Mastronarde, D. N. J. Microsc. 230, 212-217 (2008); and Mastronarde, D. N. & Held, S. R. J. Struct. Biol. 197, 102-113 (2017)] on iMac Pro and Mac Studio Ml computers (Apple, Inc.).

### Statistics

Unpaired T-tests on raw or log-transformed (viral titer) data were performed using GraphPad Prism 10 or R [The R Project for Statistical Computing. www.r-project.org/]. Statistical significance is indicated with (p=. 05-. 1), * (p<0.05), ** (p<0.01), *** (p<. 001) or (p<. 0001).

### Results/Discussion

### Cysteine in the N Linker Promotes the Formation of a Stable N-N Dimer

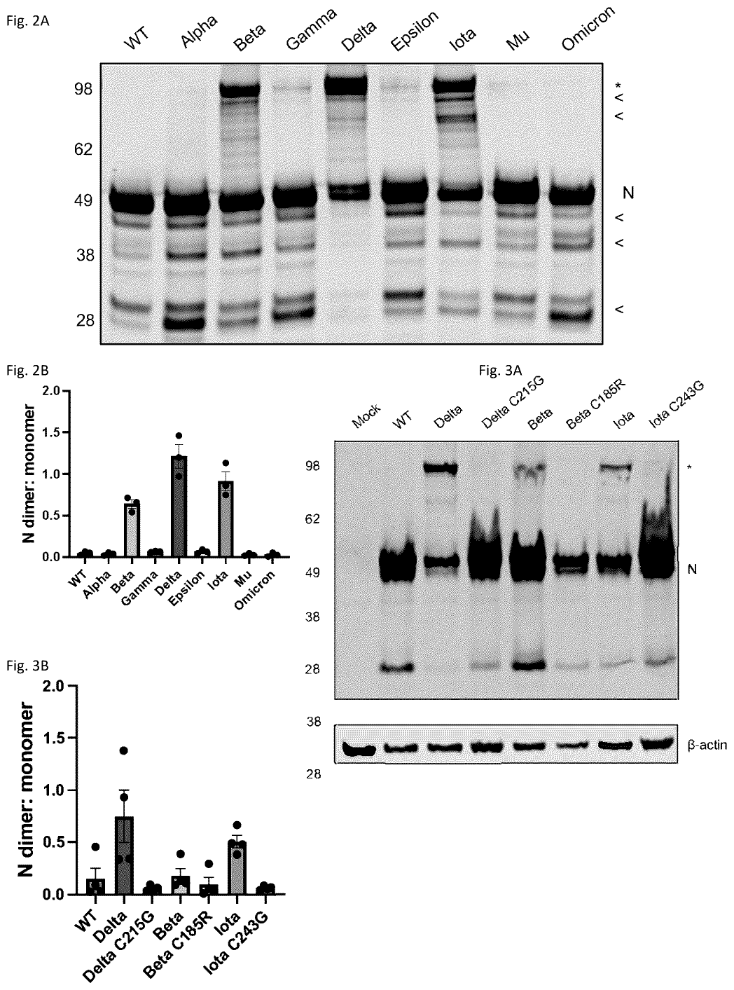

To test if these novel cysteines would make a more stable N-N dimer, the N from the panel of variant isolates was visualized by western blot under non-reducing conditions. VeroE6-TMPRSS2 cells were infected with wildtype virus (ancestral SARS-COV-2 from the WA-1 infectious clone) as well as low passage stocks of the Alpha, Beta, Gamma, Delta, Epsilon, Iota, Mu, and Omicron variants isolated from clinical samples. All viruses produced a band of the expected molecular weight (˜47 kDA) for the N monomer, and the majority also showed a series of truncation products (indicated with <) we hypothesize to be caspase cleavage products [Chu, H. et al. Nature 609, 785-792 (2022)] (FIG. 2A). Results demonstrated that the three variants that contained the novel cysteine residue (Beta, Delta, Iota) produced a second band detected at twice the molecular weight of the expected N monomer (FIG. 2A, see *). Of the three variants, Delta (G215C) produced the greatest level of dimerized-N, with Iota (G243C) and Beta (R185C) each producing slightly lesser amounts (FIG. 2B). Coronavirus N proteins typically form dimers via their dimerization domains, which is mediated by non-covalent bonds and the canonical N-dimer was not seen by SDS-PAGE gel/western blot for viruses that lacked cysteines (WT, Alpha, Gamma, Epsilon, Mu, and Omicron).

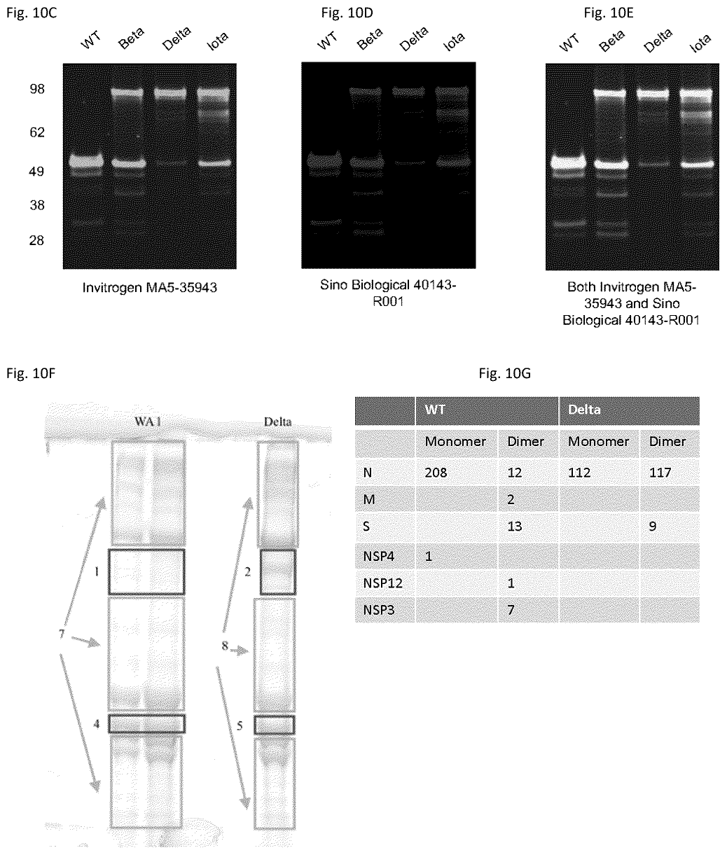

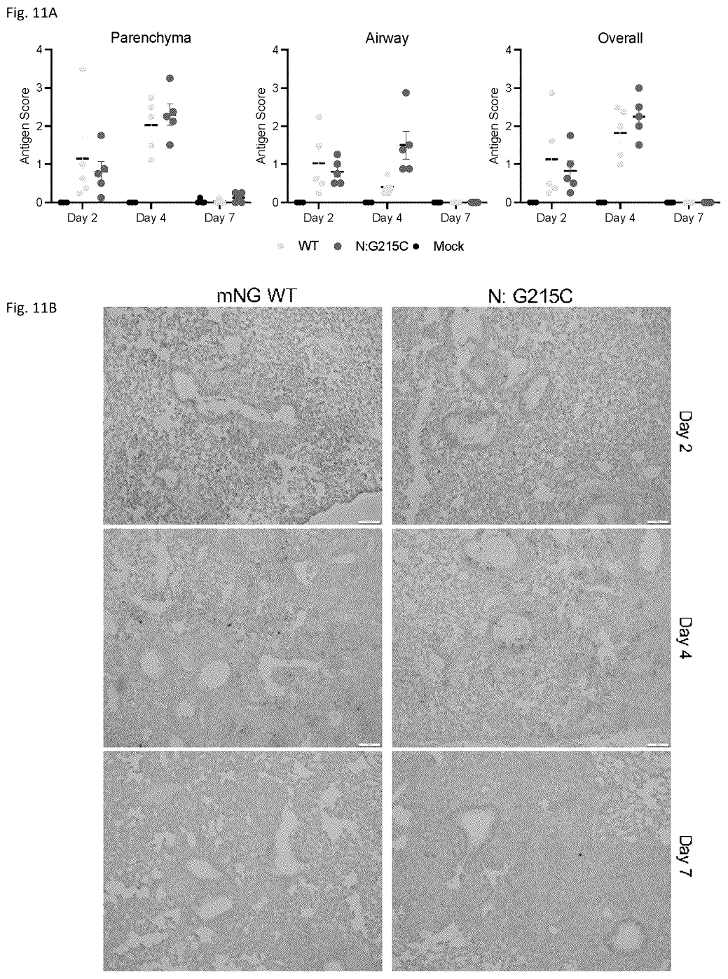

To further test whether this higher molecular weight band represented a disulfide-bonded form of N-N dimer several additional experiments were performed. First, samples were prepared under strong reducing conditions (10 mM DTT), where results showed the higher molecular weight band was eliminated (FIG. 10A). To ensure that this disulfide bond truly occurred within infected cells and was not a post-lysis artifact, lysates were harvested in the presence of n-ethylmaleimide (NEM). NEM binds irreversibly to free cysteines and prevents the formation of post-lysis disulfide bonds, and the band belonging to the putative N dimer remained visible in these conditions (FIG. 10B). To confirm the putative dimer was not an artifact of the monoclonal antibody used, lysates were probed with a second independent antibody (FIG. 10C-D). Slight differences in cleavage products were seen with the two antibodies (see *), confirming the antibodies recognized different epitopes, but the higher molecular weight band was visible in both conditions (FIG. 10C-E). To ensure the higher molecular band did not represent a disulfide bond formed between N and a cellular or viral protein of equivalent weight, N was immune-precipitated from cells infected with WT or Delta SARS-COV-2 and proteomics was performed on gel slices cut from the regions corresponding to the monomer and putative ‘dimer’ (FIG. 10F). In the Delta infected sample, most peptides detected in the ‘dimer’ gel slice corresponded to N, and roughly half the total N peptides were detected in the ‘dimer’ vs ‘monomer’ slice (FIG. 10G). Furthermore, there were no cellular peptides of similar abundance found in the ‘dimer’ slice, with the most abundant cellular protein found at >⅕^{th} the levels of N. The results indicated that the introduction of this disulfide bond promoted formation of a tetramer or higher order structure by stabilizing the bond between two non-covalently-bonded dimers.

### N Dimer Stability Dependent on Novel Cysteines in Linker Region.

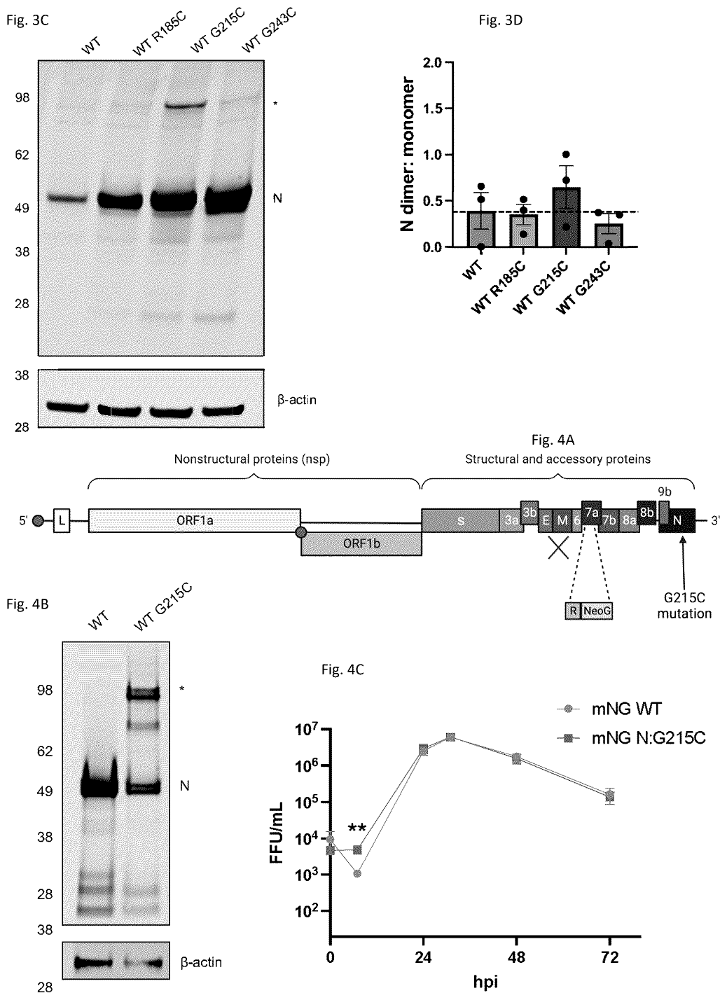

As the production of a disulfide bond in the reducing environment of the cytoplasm is unusual, studies were then performed to determine whether the formation of this stable N-N dimer required the context of infection. SARS-COV-2 replication produced many membranous compartments with limited cytoplasmic access that could shield N during authentic infection. Plasmid expression constructs of the WT, Delta, Beta, and Iota nucleocapsid sequences were created and transfected into HEK-293T cells, in conjunction with constructs where each cysteine was mutated back to the canonical residue in the WT sequence (Delta C215G, Beta C185R, Iota C243G). Although all three variant constructs (Delta, Beta, Iota) were capable of producing stably dimerized N (FIG. 3A), Delta produced this product to the highest levels while the Beta construct did not consistently form visible, stable dimer (FIG. 3B). In each case, when the cysteine was reverted to its original amino acid the stabilized dimer was not made (FIG. 3A-B). This data indicated that, at least in the case of Delta and Iota, the presence of other viral proteins/RNA and the formation of double membrane vesicles (DMVs) are not required for stably dimerized N formation and the presence of a cysteine at 215/243 in the Delta/Iota backgrounds is sufficient.

### Impact of the N: G215C Mutation on Viral Growth In Vitro and In Vivo

Studies were then performed to examine the impact of the G215C mutation on viral growth kinetics. Because WT SARS-COV-2 (WA-1) does not produce the stably dimerized N in infection (FIG. 2A), experiments were performed to investigate if the introduction of a cysteine at 185, 215 or 243 (Beta, Delta, Iota) was sufficient to mediate stable dimer formation in a WT background. Using plasmid constructs, HEK-293T cells were transfected with either WT N, or WT N containing a single point mutation (R185C, G215C, and G234C). the term “point mutation” when used in relation to a protein or amino acid sequence means an amino acid substitution. For example, a single point mutation in a protein means a single amino acid substitution. When visualized via western blot in non-reducing conditions, only the construct which contained the Delta G215C mutation could produce the stabilized dimer, suggesting that this mutation is both necessary and sufficient for stable dimer formation (FIG. 3C-D).

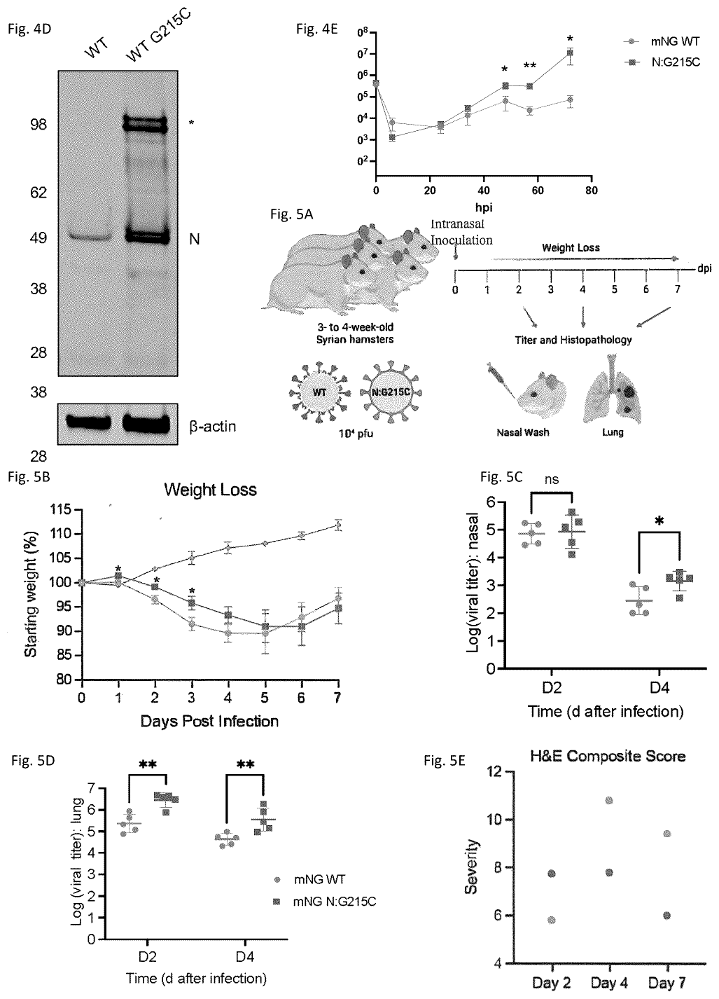

As inserting the G215C mutation in the WT background was sufficient to confer dimer formation, a well-established reverse genetics system [Xie, X. et al. Cell Host Microbe 27, 841-848.e3 (2020)] was used to rescue an infectious clone containing the G215C nucleocapsid mutation (N: G215C) in the SARS-COV-2 WA1 backbone (FIG. 4A). A neon-green reporter virus was used in the WA-1 background (mNG SARS-COV-2), which was genetically identical to WA-1 except that ORF7 had been replaced with a neon green fluorescent reporter. Notably, mNG SARS-COV-2 is attenuated compared to the parental WA-1, due to the replacement of ORF7a with the mNG reporter [Johnson, B. A. et al., PLOS Pathog. 18, e1010627 (2022)]. The N: G215C virus produced the stable N dimer in infected Vero-TMPRSS2 cells under non-reducing conditions (FIG. 4B). In a multi-cycle growth curve, the WT and N: G215C viruses grew to identical peak titers, with very similar growth kinetics. It was noted that at the earliest timepoint (7 hpi), the WT virus showed an ˜1 log drop in viral titer (representing the loss in infectivity of the original inoculum), while the N: G215C virus did not display such a drop (FIG. 4C), though the biological significance (if any) of this observation is unclear.

Next, as VeroE6 cells are not a physiologically relevant target for SARS-COV-2 infection, viral growth kinetics were examined in primary differentiated human bronchial epithelial cells (HBECs), grown in transwells on an air-liquid interface (ALI). The N: G215C virus produced the stable N dimer in infected HBECs when harvested under non-reducing conditions (FIG. 4D). Notably, in multi-cycle growth curves the mNG N: G215C virus had improved growth kinetics, with a peak titer more than 100 times greater than the mNG WT virus (FIG. 4E). This data indicated the stably dimerized form of N conveyed a particular advantage to viral replication in primary differentiated human bronchial cells.

Next, studies were performed to determine the effect of the N: G215C mutation in vivo in the Syrian Golden Hamster model of SARS-COV-2 infection. Three to four week-old male hamsters were inoculated intranasally with either PBS (mock), 10⁴ PFU of the WT neon green reporter SARS-COV-2 (mNG WT) or 10⁴ PFU of the neon green reporter SARS-COV-2 containing the N: G215C mutation (N: G215C). Animals were monitored for weight gain/loss daily for seven days, and cohorts of five animals underwent nasal washing followed by euthanasia to obtain tissues to determine viral loads in the lung at both day 2 and day 4 (FIG. 5A). On day seven, surviving animals were euthanized and tissue collected for virological and histopathological analysis. While animals infected with the N: G215C mutation showed significant weight loss, relative to control WT virus, the kinetics were delayed with peak disease achieved at day 5-6 post infection, 1-2 days after WT infection (FIG. 5B). Strikingly, despite delayed weight loss, viral replication was increased with the G215C mutation. Modest but significant increases in viral titers were observed in the nasal washes at day 4, and a sustained 10-fold increase over WT titers in the lungs throughout the infection (FIG. 5C-D). Together, despite the kinetic delay, the N: G215C mutant caused similar overall weight loss and augmented viral replication.

Examining histopathology, the N: G215C mutant had modest changes in antigen staining, but increased infiltration and damage relative to WT control virus. At day 2, both WT and N: G215C had similar antigen distribution and scores (FIG. 11A-B). By day 4, N: G215 had a modest increase in overall antigen staining mostly driven by significant differences in airway staining. Viral staining was cleared in both WT and mutant infected animals by day 7 post infection. Examining immune infiltration and damage, lesions were of similar composition and size at day 2 for both groups, but more severe in WT animals (FIG. 5E-F). However, at day 4, N: G215C had increased infiltration and damage compared to WT infected animals. While interstitial pneumonia, bronchiolitis, periarterial edema was common in both groups, N: G215C infected mice were consistently observed to have epithelial cytopathology and subendothelial mononuclear cells. Similarly at day 7, N: G215C maintained evidence of significant damage with continued peribrochiolitis and epithelial cytopathology; in contrast, WT infected hamsters had reduced overall damage scores. Together, the results demonstrate that despite similar weight loss, the N: G215C mutant infected animals have increased viral antigen accumulation and damage in the lung as compared to control.

### Wildtype SARS-COV-2 Containing the G215C Mutation Packages More Nucleocapsid Protein Per Virion and Displays Oblong Morphology

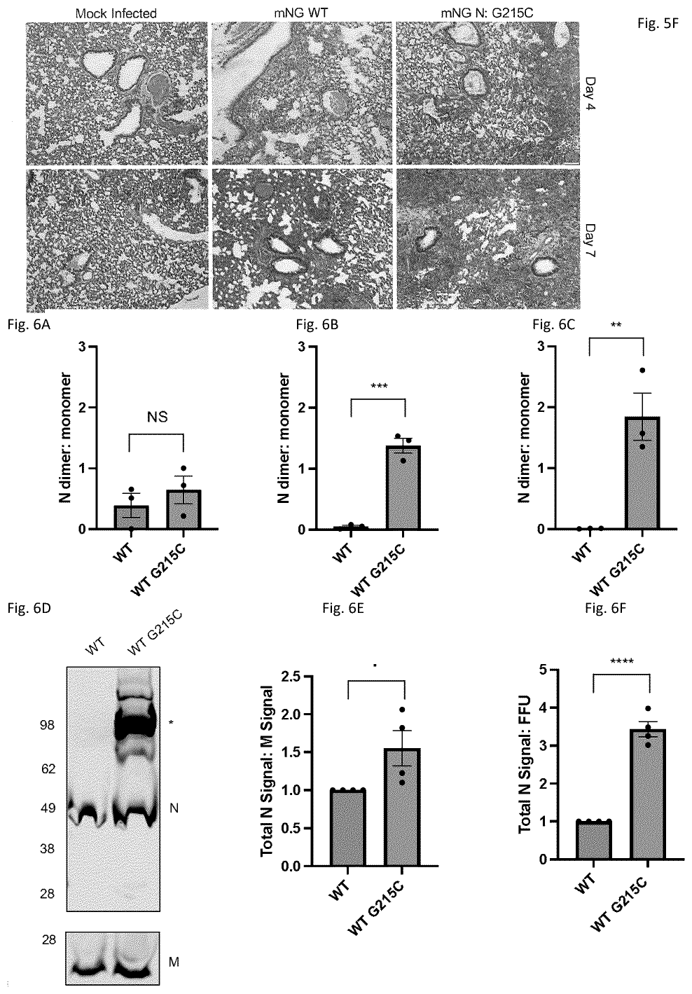

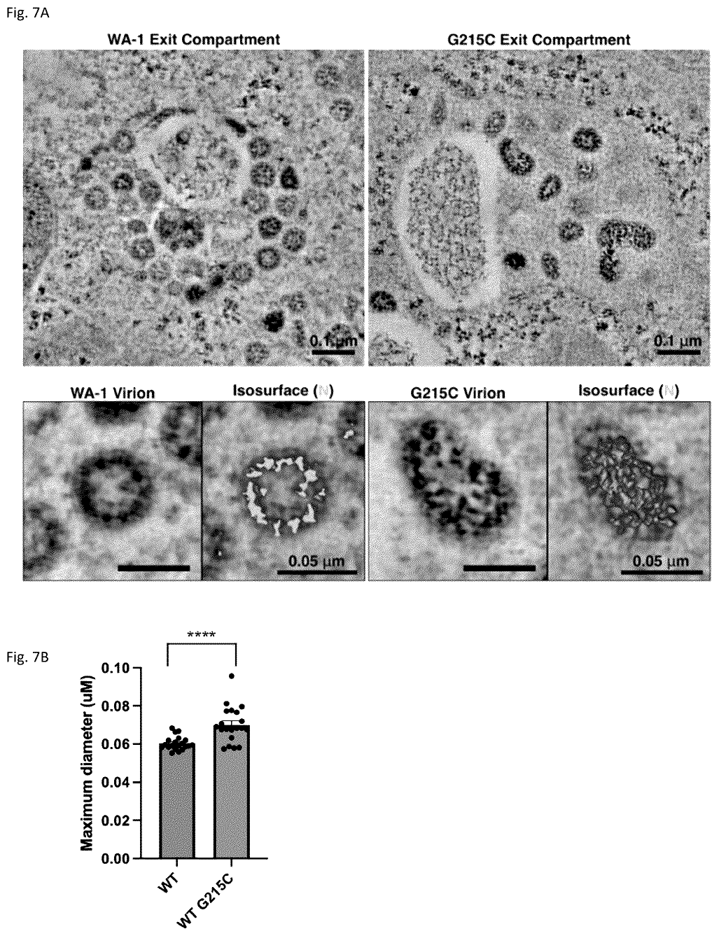

The SARS-COV-2 nucleocapsid, like nucleocapsids of other Betacoronaviruses, is a highly multifunctional protein. Like other coronavirus nucleocapsids, it is thought to play key roles in the packaging of viral RNA [Jack, A. et al. PLOS Biol. 19, e3001425 (2021)], the production of viral RNA through interactions with the replication-transcription complex [Zúñiga, S. et al. J. Virol. 84, 2169-2175 (2010); Almazán, F., Galán, C. & Enjuanes, L. J. Virol. 78, 12683-12688 (2004); Schelle, B. et al., J. Virol. 79, 6620-6630 (2005); and Thiel, V., et al., J. Virol. 75, 6676-6681 (2001)], and the antagonism of the innate immune response [Aloise, C. et al. PLOS Pathog. 19, e1011582 (2023); LeBlanc, K. et al. Microbiol. Spectr. 11, e00994-23 (2023); and Liu, H. et al. J. Virol. 96, e00412-22 (2022)]. To better understand why the N: G215C mutation was important at a molecular level, studies were performed to identify where in the viral life-cycle the stably dimerized form of N was observed. The stable N-dimer was observed in lysates of cells infected with the Beta, Delta, and Iota variants (FIG. 2A), though cells transfected with the Delta (and to lesser extent Iota) nucleocapsids were able to form the durable N-dimer even in the absence of other viral machinery (FIG. 3B). Because a gradient of dimer formation had been observed, depending on where the cysteine mutation was located (215>243>185; FIG. 2B, 3B), studies were done to explore whether the stable G215C N-dimer was found at highest levels in transfected cells, infected cells, or in concentrated extracellular virions. Accordingly, the studies included measuring the ratio of N dimer: N monomer visible on a western blot of samples collected under non-reducing conditions from transfected HEK-293T cells, infected Vero-TMPRSS2 cells, or extracellular virus concentrated by polyethylene glycol (PEG) precipitation (FIG. 6A-C). Interestingly, it was noted that the dimer was enriched in extracellular virus (FIG. 6C-D), though infection (vs transfection in isolation) also appeared to promote formation of the stably dimerized N (FIG. 6B vs 6A).

As this enrichment of dimerized N in virions suggested a potential role in encapsidation, studies were performed to compare the incorporation of total levels of N to levels of M in the WT vs N: G215C viruses (FIG. 6D-E). Finally, the levels of N were compared to the number of infectious focus forming units (FFU) for the two viruses (FIG. 6F). In both cases the N: G215C virus appeared to over-incorporate N in virions, compared to another structural protein (M) or infectious units, suggesting that the stably dimerized N had increased encapsidation activity compared to the WT nucleocapsid protein.

In order to examine these apparent changes in encapsidation and packaging on a single virion level, thin section transmission electron microscopy (TEM) was performed on Vero-TMPRSS2 cells that were infected with the WT or N: G215C virus. The study included examination of the morphology of mature virions that had completed budding into intracellular compartments and were no longer attached to the cellular membrane, assessing virion shape as well as the amount and arrangement of internal nucleocapsid structures inside individual virions (FIG. 7). The WT virions were largely round and ˜60 μM in diameter, with electron dense complexes likely representing ribonucleoprotein (N+RNA) complexes packed inside (FIG. 7A). The N: G215C virus produced some spherical particles similar to WT virions, but it also produced a substantial fraction of virions that displayed oblong or elongated morphologies and were larger than the WT virions in circumference (FIG. 7A-B). These virions also appeared to package more ribonucleoprotein complex structures than the smaller spherical virions, in agreement with previous biochemical analysis of bulk virions.

### DISCUSSION

In experiments such as those disclosed herein, is study, the role of unique cysteine residues that were inserted into the SARS-COV-2 nucleocapsid linker domain was investigated in several Variants of Concern. Results demonstrate that these cysteines formed stable disulfide bonds that linked nucleocapsid proteins together, outside of the canonical dimerization domain. Sequencing data from public health surveillance efforts suggested that the insertion of a cysteine at position 214 (Lambda) and 215 (Delta) were maintained in these two lineages (FIG. 1), and the experimental data supported the observation that the G215C mutation is beneficial to the virus in vitro (FIG. 4E) and in vivo (FIG. 5B-C). Although Betacoronaviruses are known to form dimers, this process is generally thought to occur via a dimerization domain in the C terminus of the protein and importantly is not normally the result of a disulfide bond. Due to the location of the two conserved cysteine residues (214 & 215) in the center of the flexible N linker, and prior data suggesting the linker can play a role in oligomerization [Zhao, H. et al. PNAS Nexus 1, pgac049 (2022) and Ye, Q., et al., Protein Sci. 29, 1890-1901 (2020)], studies such as those disclosed herein were performed and provided evidence that these mutations form disulfide bonds between pairs of N-N dimers and mediate higher order N oligomerization (FIG. 8). Increasing the affinity of the inter-linker interactions that mediate oligomerization could have the effect of shifting the balance to N towards higher levels of oligomerization.

N is a highly multifunctional protein, and it is likely that some of the regulation of its multiple activities depends on whether it is in the monomeric or oligomerized form. It is thought that phosphorylation of N, specifically within the SR region of the linker, acts as a biological ‘switch’ and mediates the switch between genome packaging/assembly and N's intracellular roles (including binding NSP3) [Johnson, B. A. et al. PLOS Pathog. 18, e1010627 (2022); Cubuk, J. et al. Nat. Commun. 12, 1936 (2021); Lu, S. et al. Nat. Commun. 12, 502 (2021); and Carlson, C. R. et al. Mol. Cell 80, 1092-1103.e4 (2020)]. The G215C Delta mutation lies only slightly upstream of key mutations at 203/204 seen in multiple VOCs that modulate viral pathogenicity [Johnson, B. A. et al. PLOS Pathog. 18, e1010627 (2022)]. It is possible that oligomerization status and phosphorylation states play interacting roles, and that steric considerations affect the ability of cellular kinases to access these residues in the oligomerized state. Nucleocapsid in mature virions is known to be hypo-phosphorylated in the SR region that encompasses both the 203/204 and 215 mutations, in contrast to the intracellular pool which is hyper-phosphorylated [Wu, C.-H. et al., J. Biol. Chem. 284, 5229-5239 (2009) and Wu, C.-H., et al., Cell Host Microbe 16, 462-472 (2014)]. Data resulting from studies presented herein indicates that the stably dimerized form of N is packaged at higher levels into mature virions, which may be partially due to a decreased ability of cellular kinases to access the key phosphorylation sites.

One of the key roles of the nucleocapsid is to encapsidate the viral RNA during the process of packaging the full-length viral genome into newly forming virions, helping to chaperone and protect the viral genome in its transition from the producer to new target cell. It is known that the nucleocapsid bound to the viral genome is generally in higher order structures (˜14-20 nm, likely composed of 12 copies of N), around which the viral RNA is wrapped (similar to beads on a string) [Yao, H. et al. Cell 183, 730-738.e13 (2020); Klein, S. et al. Nat. Commun. 11, 5885 (2020); Bracquemond, D. & Muriaux, D. mBio 12, 10.1128/mbio.02371-21 (2021)]. Data resulting from studies such as those presented herein support this framework, as the stably dimerized form of N is seen preferentially in free virus (FIG. 6C) compared to transfected or infected cells (FIG. 6A-B). Increasing the stability of nucleocapsid oligomers could increase the rate or efficiency of viral encapsidation, which fits with the observation that the G215C mutation increases the amount of nucleocapsid packaged into virions, compared to infectious viral units or other viral structural proteins (FIG. 6E-F).

Although SARS-COV-2 virions lack the rigid organization of a virus with a defined icosahedral capsid, a 50-300% increase of nucleocapsid would seem difficult to accommodate within the standard shape a WT virion. Indeed, the results of studies such as those presented herein, indicate that, for at least a portion of particles, shifting the oligomerization status by introducing the G215C mutation results in particles that are elongated compared to WT virus (FIG. 7). This finding is particularly interesting in light of recent observations that the virions of a clinical Delta isolate retain the spherical shape and radius size found in the ancestral SARS-CoV-2 lineage [Ke, Z. et al. 2023.12.21.572824 Preprint at doi.org/10.1101/2023.12.21.572824 (2023)], suggesting that additional mutations in the Delta lineage have the effect of counteracting the phenotype we observe with the G215C virus.

Relatedly, one of the critical unanswered questions of this study is why no Omicron sub-lineages have possessed such a cysteine in the linker region of the nucleocapsid. It is clear from human transmission data, as well as the clear beneficial effect in vitro and in vivo (FIG. 4E, FIG. 5B-C) of the G215C mutation that a cysteine in this region of the nucleocapsid is beneficial. Differing stability of dimer formation at different positions however (greatest at 215>243>185; FIG. 2B and FIG. 3B) suggests that the 215 location is preferred over bond formation closer to either the RNA-binding or dimerization-domains. Furthermore, while the mutations at 214 and 215 were evolutionarily maintained in the Lambda and Delta lineages, the mutations at 243 and 185 observed in the Iota and Beta stocks were not maintained in these lineages. It is possible that there has not yet been sufficient time for a cysteine-causing mutation to arise at the 214/215 residue of nucleocapsid, and this mutation could occur in a future variant of concern (in the context of omicron or an as-of-yet unknown future variant). The present data suggests this mutation would be likely to increase viral titers significantly, and mutations in this region should be monitored closely for their public health implications. It is also possible that there are epistatic interactions that mediate the beneficial effect of the G215C mutation that are not present in the Omicron background. While the G215C mutation was clearly beneficial for Delta (FIG. 1), as well as ancestral SARS-COV-2 in the absence of any other complementary mutations (FIG. 4E, 5B-C), examination of the Omicron background is helpful to understand the effect this mutation would have on currently circulating virus lineages.

Curiously, the mutations observe in this study (particularly 215) lie on the binding interface between the N linker and the NSP3 Ubl1 domain [Bessa, L. M. et al., Sci. Adv. 8, eabm4034 (2022)], and it is believed that stably dimerized N would have a reduced ability to interact with NSP3. There is clear evidence that the interaction between N and NSP3 at this binding interface is important for coronavirus biology, and mismatches between the N linker region and the NSP3 Ubl1 domain severely attenuate viral titers or block viral rescue [Hurst, K. R., et al., J. Virol. 87, 9159-9172 (2013) and Hurst, K. R., et al., J. Virol. 84, 10276-10288 (2010)]. The function of the N-NSP3 interaction has not been fully resolved, though it has been suggested to play critical roles in either tethering the incoming viral genome to the replication-transcription complex, or delivering the viral genome to the DMV, to ensure successful early replication [Hurst, K. R., et al., J. Virol. 87, 9159-9172 (2013); Hurst, K. R., et al., J. Virol. 84, 10276-10288 (2010); and Koetzner, C. A., et al., Virology 567, 1-14 (2022)]. N is known to play a key role early in infection, as the gRNA of coronaviruses (unlike nearly all other positive strand RNA viruses) is only minimally infectious in the absence of N protein, suggesting that this N-NSP3 interaction is likely key for efficient replication or transcription of viral RNA [Yount, B. et al. Proc. Natl. Acad. Sci. 100, 12995-13000 (2003); Yount, B., et al., J. Virol. 76, 11065-11078 (2002); Yount, B., et al., J. Virol. 74, 10600-10611 (2000); Coley, S. E. et al. J. Virol. 79, 3097-3106 (2005); and Casais, R., et al., J. Virol. 75, 12359-12369 (2001)].

In addition to its role in the replicase, NSP3 forms a key portion of the pore connecting the cytoplasm to the interior of the double membrane vesicle (DMV) where viral transcription occurs [Snijder, E. J. et al. PLOS Biol. 18, e3000715 (2020) and Wolff, G. et al., Science 369, 1395-1398 (2020)]. NSP3 comprises the outermost (cytoplasmic facing) layer of the DMV, with the Ubl1 domain at the very tip of the ‘prongs’ that extend outwards from the pore into the cytoplasm [Wolff, G. et al., Science 369, 1395-1398 (2020)]. Recent structural work proposed that the interaction between the linker region of N and the Ubl1 domain of NSP3 helps to mediate a condensation of the nucleocapsid protein prior to encapsidation [Bessa, L. M. et al., Sci. Adv. 8, eabm4034 (2022)]. In addition, this interaction is proposed to tether nucleocapsid molecules in the immediate vicinity of the DMV exit, ensuring that viral genomes are immediately coated with N as they are extruded out from the DMV pore [Bessa, L. M. et al. Sci. Adv. 8, eabm4034 (2022)]. The mutations and viruses characterized in studies presented herein are useful reagents for future studies studying the functional implications of the N-NSP3 interaction.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of increasing antigenicity of a viral immunization preparation, the method comprising: including in the viral immunization preparation a modified viral nucleocapsid (N) protein or a sequence encoding the modified viral N protein, wherein the modified viral N protein comprises an amino acid substitution in a linker region of a viral N protein, and wherein the expressed modified viral N protein in the viral immunization preparation increases a level of N-N dimers in the viral immunization preparation compared to a control level of N-N dimers and increases the level of antigenicity of the viral immunization preparation compared to a control level of antigenicity.

2-5. (canceled)

6. The method of claim 1, wherein the viral N protein is a coronavirus N protein.

7-10. (canceled)

11. The method of claim 1, wherein the viral immunization preparation further comprises at least one additional independently selected viral protein, optionally wherein the at least one additional independently selected viral protein is one of more of a viral spike protein, a viral capsid protein, a viral envelope protein, and a viral membrane protein.

12-14. (canceled)

15. The method of claim 1, wherein the amino acid sequence of the viral N protein is SEQ ID NO: 1 or a functional variant of SEQ ID NO: 1 comprising one or more amino acid substitution mutations comprising a glycine→cysteine (G→C) substitution and an arginine→cystine (R→C) substitution in the viral N protein, optionally wherein the one or more amino acid substitutions is at (i) G215 of SEQ ID NO: 1, or at a glycine in the functional variant of SEQ ID NO: 1 at a position that corresponds to the G215 in SEQ ID NO: 1; (ii) G243 of SEQ ID NO: 1, or at a glycine in the functional variant of SEQ ID NO: 1 at a position that corresponds to the G243 in SEQ ID NO: 1; or (iii) R185 of SEQ ID NO: 1, or at an arginine in the functional variant of SEQ ID NO: 1 at a position that corresponds to the R185 in SEQ ID NO: 1.

16. (canceled)

17. The method of claim 1, wherein the amino acid sequence of the modified N protein is (i) SEQ ID NO: 2, or a functional variant of SEQ ID NO: 2, (ii) SEQ ID NO: 3, or a functional variant of SEQ ID NO: 3, (iii) SEQ ID NO: 4, or a functional variant of SEQ ID NO: 4; (iv) SEQ ID NO: 5 or a functional variant of SEQ ID NO: 5; (v) SEQ ID NO: 6 or a functional variant of SEQ ID NO: 6; (vi) SEQ ID NO: 7 or a functional variant of SEQ ID NO: 7; or (vii) SEQ ID NO: 8 or a functional variant of SEQ ID NO: 8.

18-25. (canceled)

26. A method of increasing a level of N-N dimers in a virus particle, the method comprising including in the virus particle, a modified viral nucleocapsid (N) protein comprising an amino acid substitution in a linker region of a viral N protein, wherein the presence of the amino acid substitution increases a level of N-N dimers in the viral particle compared to a control level of N-N dimers, optionally wherein the control level of N-N dimers is a level of N-N dimers in a substantially similar viral particle in the absence of the modified viral N protein.

27. (canceled)

28. The method of claim 26, wherein the viral N protein is a coronavirus N protein.

29-33. (canceled)

34. The method of claim 26, wherein the amino acid sequence of the modified viral N protein is SEQ ID NO: 1 or a functional variant of SEQ ID NO: 1 comprising one or more amino acid substitution comprising a glycine→cysteine (G→C) substitution and an arginine→cystine (R→C) substitution in the viral N protein, optionally wherein the one or more amino acid substitutions is at (i) G215 of SEQ ID NO: 1, or at a glycine in the functional variant of SEQ ID NO: 1 at a position that corresponds to the G215 in SEQ ID NO: 1; (ii) G243 of SEQ ID NO: 1, or at a glycine in the functional variant of SEQ ID NO: 1 at a position that corresponds to the G243 in SEQ ID NO: 1; or (iii) R185 of SEQ ID NO: 1, or at an arginine in the functional variant of SEQ ID NO: 1 at a position that corresponds to the R185 in SEQ ID NO: 1.

35. (canceled)

36. The method of claim 26, wherein the amino acid sequence of the modified N protein is (i) SEQ ID NO: 2, or a functional variant of SEQ ID NO: 2; (ii) SEQ ID NO: 3, or a functional variant of SEQ ID NO: 3; (iii) SEQ ID NO: 4, or a functional variant of SEQ ID NO: 4; (iv) SEQ ID NO: 5 or a functional variant of SEQ ID NO: 5; (v) SEQ ID NO: 6 or a functional variant of SEQ ID NO: 6; (vi) SEQ ID NO: 7 or a functional variant of SEQ ID NO: 7; or (vii) SEQ ID NO: 8 or a functional variant of SEQ ID NO: 8.

37-44. (canceled)

45. A composition comprising a modified viral nucleocapsid (N) protein or a functional fragment thereof, wherein the modified viral N protein and the functional fragment thereof comprise an amino acid substitution in a linker region of the viral N protein, optionally wherein the viral N protein is a coronavirus N protein.

46-49. (canceled)

50. The composition of claim 45, wherein the amino acid sequence of the viral N protein is SEQ ID NO: 1, or a functional variant of SEQ ID NO: 1, and optionally wherein the amino acid substitution is a glycine→cysteine (G→C) substitution or an arginine→cystine (R→C) substitution in the viral N protein.

51. (canceled)

52. The composition of claim 45, wherein the amino acid substitution is at (i) amino acid G215 of SEQ ID NO: 1, or at a glycine in the functional variant of SEQ ID NO: 1 at a position that corresponds to the G215 in SEQ ID NO: 1; (ii) G243 of SEQ ID NO: 1, or at a glycine in the functional variant of SEQ ID NO: 1 at a position that corresponds to the G243 in SEQ ID NO: 1 or (iii) R185 of SEQ ID NO: 1, or at an arginine in the functional variant of SEQ ID NO: 1 at a position that corresponds to the R185 in SEQ ID NO: 1.

53-56. (canceled)

57. The composition of claim 45, wherein the amino acid sequence of the modified N protein is (i) SEQ ID NO: 2, or a functional variant of SEQ ID NO: 2, (ii) SEQ ID NO: 3, or a functional variant of SEQ ID NO: 3, (iii) SEQ ID NO: 4, or a functional variant of SEQ ID NO: 4; (iv) SEQ ID NO: 5 or a functional variant of SEQ ID NO: 5; (v) SEQ ID NO: 6 or a functional variant of SEQ ID NO: 6; (vi) SEQ ID NO: 7 or a functional variant of SEQ ID NO: 7; or (vii) SEQ ID NO: 8 or a functional variant of SEQ ID NO: 8.

58-61. (canceled)

62. A fusion protein comprising a modified N protein composition of claim 45.

63. A cell comprising the fusion protein of claim 62.

64-65. (canceled)

66. A viral immunization preparation comprising the modified N protein composition of claim 45.

67. A viral particle comprising the modified N protein composition or a functional fragment thereof of claim 45.

68. (canceled)

69. A viral immunization preparation comprising the viral particle of claim 67.

70. A polynucleotide (DNA) molecule encoding the modified N protein composition of claim 45.

71-73. (canceled)

74. A viral immunization preparation comprising the polynucleotide (DNA) molecule of claim 70.

75-97. (canceled)


SEQUENCE LISTING (61 sequences)

SEQ ID NO: 1 - protein, 419 aa, Coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 419 aa, Coronavirus
MSDNGPQNQRNAPRITFGGPSDSTGSNQNGERSGARSKQRRPQGLPNNTASWFTALTQHGKEGLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSMGTSPARMAGNGCDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQA

SEQ ID NO: 3 - protein, 419 aa, Coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSCSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 4 - protein, 419 aa, Coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTLGSSRGTSPARMAGNGGDAALALLLLDRLNQLESKISGKGQQQQCQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSCSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGCDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 7 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQCQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 8 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNCGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 74 aa, synthetic construct
GSRGGSQASSRSSSRSRNSSRNSTPGSSRGTSPARMAGNGGDAALALLLLDRLNQLESKMSGKGQQQQGQTVTK

SEQ ID NO: 10 - DNA, 222 bp, synthetic construct
  source 1..222: mol_type = other DNA
gggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaag

SEQ ID NO: 11 - protein, 74 aa, synthetic construct
GSRGGSQASSRSSSRSRNSSRNSTPGSSMGTSPARMAGNGCDAALALLLLDRLNQLESKMSGKGQQQQGQTVTK

SEQ ID NO: 12 - DNA, 222 bp, synthetic construct
  source 1..222: mol_type = other DNA
gggagcagaggcggcagtcaagcctcttctcgttcctcatcacgtagtcgcaacagttcaagaaattcaactccaggcagcagtatgggaacttctcctgctagaatggctggcaatggctgtgatgctgctcttgctttgctgctgcttgacagattgaaccagcttgagagcaaaatgtctggtaaaggccaacaacaacaaggccaaactgtcactaag

SEQ ID NO: 13 - protein, 74 aa, synthetic construct
GSRGGSQASSCSSSRSRNSSRNSTPGSSRGISPARMAGNGGDAALALLLLDRLNQLESKMSGKGQQQQGQTVTK

SEQ ID NO: 14 - DNA, 222 bp, synthetic construct
  source 1..222: mol_type = other DNA
gggagcagaggcggcagtcaagcctcttcttgttcctcatcacgtagtcgcaacagttcaagaaattcaactccaggcagcagtaggggaatttctcctgctagaatggctggcaatggcggtgatgctgctcttgctttgctgctgcttgacagattgaaccagcttgagagcaaaatgtctggtaaaggccaacaacaacaaggccaaactgtcactaag

SEQ ID NO: 15 - protein, 73 aa, synthetic construct
SRGGSQASSRSSSRSRNSSRNSTLGSSRGTSPARMAGNGGDAALALLLLDRLNQLESKISGKGQQQQCQTVTK

SEQ ID NO: 16 - DNA, 219 bp, synthetic construct
  source 1..219: mol_type = other DNA
agcagaggcggcagtcaagcctcttctcgttcctcatcacgtagtcgcaacagttcaagaaattcaactctaggcagcagtaggggaacttctcctgctagaatggctggcaatggcggtgatgctgctcttgctttgctgctgcttgacagattgaaccagcttgagagcaaaatatctggtaaaggccaacaacaacaatgccaaactgtcactaag

SEQ ID NO: 17 - DNA, 1257 bp, Coronavirus
  source 1..1257: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 18 - DNA, 1257 bp, Coronavirus
  source 1..1257: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaaggccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctatgcagaagggagcagaggcggcagtcaagcctcttctcgttcctcatcacgtagtcgcaacagttcaagaaattcaactccaggcagcagtatgggaacttctcctgctagaatggctggcaatggctgtgatgctgctcttgctttgctgctgcttgacagattgaaccagcttgagagcaaaatgtctggtaaaggccaacaacaacaaggccaaactgtcactaagaaatctgctgctgaggcttctaagaagcctcgacaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggcttatgaaactcaagccttaccgcagagacagaagaaacagcaaactgtgactcttcttcctgctgcagatttggatgatttctccaaacaattgcaacaatccatgagtagtgctgactcaactcaggcc

SEQ ID NO: 19 - DNA, 1257 bp, Coronavirus
  source 1..1257: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttcttgttcctcatcacgtagtcgcaacagttcaagaaattcaactccaggcagcagtaggggaatttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 20 - DNA, 1257 bp, Coronavirus
  source 1..1257: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactctaggcagcagtaggggaacttctcctgctagaatggctggcaatggcggtgatgctgctcttgctttgctgctgcttgacagattgaaccagcttgagagcaaaatatctggtaaaggccaacaacaacaatgccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 21 - DNA, 1257 bp, synthetic construct
  source 1..1257: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttcttgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 22 - DNA, 1257 bp, synthetic construct
  source 1..1257: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggctgtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 23 - DNA, 1257 bp, synthetic construct
  source 1..1257: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaatgtcaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 24 - DNA, 1260 bp, synthetic construct
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaattgcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 25 - protein, 190 aa, synthetic construct
GSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFP

SEQ ID NO: 26 - DNA, 570 bp, synthetic construct
  source 1..570: mol_type = other DNA
gggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattccca

SEQ ID NO: 27 - protein, 374 aa, synthetic construct
PNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 28 - DNA, 1125 bp, synthetic construct
  source 1..1125: mol_type = other DNA
cccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 29 - protein, 364 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFP

SEQ ID NO: 30 - DNA, 1092 bp, synthetic construct
  source 1..1092: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattccca

SEQ ID NO: 31 - protein, 190 aa, synthetic construct
SRGGSQASSRSSSRSRNSSRNSTPGSSMGTSPARMAGNGCDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 32 - protein, 269 aa, synthetic construct
GDGKMKDLSPRWYFYYLGTGPEAGLPYGANKDGIIWVATEGALNTPKDHIGTRNPANNAAIVLQLPQGTTLPKGFYAEGSRGGSQASSCSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 33 - protein, 214 aa, synthetic construct
SRGGSQASSRSSSRSRNSSRNSTLGSSRGTSPARMAGNGGDAALALLLLDRLNQLESKISGKGQQQQCQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQ

SEQ ID NO: 34 - protein, 215 aa, synthetic construct
PANNAAIVLQLPQGTTLPKGFYAEGSRGGSQASSCSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 35 - protein, 193 aa, synthetic construct
SRGGSQASSRSSSRSRNSSRNSTPGSSRGTSPARMAGNGCDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEP

SEQ ID NO: 36 - protein, 371 aa, synthetic construct
NQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQCQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPA

SEQ ID NO: 37 - protein, 190 aa, synthetic construct
SRGGSQASSRSSSRSRNSSRNSTPGSSRGTSPARMAGNCGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 38 - protein, 73 aa, Coronavirus
SRGGSQASSRSSSRSRNSSRNSTPGSSRGTSPARMAGNGGDAALALLLLDRLNQLESKMSGKGQQQQGQTVTK

SEQ ID NO: 39 - protein, 73 aa, Coronavirus
SRGGSQASSCSSSRSRNSSRNSTPGSSRGISPARMAGNGGDAALALLLLDRLNQLESKMSGKGQQQQGQTVTK

SEQ ID NO: 40 - protein, 73 aa, Coronavirus
SRGGSQASSRSSSRSRNSSRNSTPGSSMGTSPARMAGNGCDAALALLLLDRLNQLESKMSGKGQQQQGQTVTK

SEQ ID NO: 41 - protein, 73 aa, Coronavirus; identical to SEQ ID NO: 15

SEQ ID NO: 42 - protein, 73 aa, Coronavirus
SRGGSQASSRSSSRSRGNSRNSTPGSSRGNSPARMASGGGETALALLLLDRLNQLESKVSGKGQQQQGQTVTK

SEQ ID NO: 43 - protein, 76 aa, Coronavirus
TGGNSQSSSRASSVSRNSSRSSSQGSRSGNSTRGTSPGPSGIGAVGGDLLYLDLLNRLQALESGKVKQSQPKVITK

SEQ ID NO: 44 - protein, 103 aa, NL63-related bat coronavirus strain BtKYNL63-9b
EPKFSIALPPELSVVEFEDRSNNSSRASSRSSTRNNSRDSSRSTSRQQSRTRSDSNQSSSDLVAAVNLALKNLGFDNQSKSPSSSGTSTPKKPNKPLSQPRAD

SEQ ID NO: 45 - protein, 85 aa, Coronavirus
DLKFDGNVPNDFKLEQNVGSRNNSRSRSRGRSKSNNRSNNNNSNSGDIATAVVAALAQMGFAPKDTQKNKSRSKSRDRSKSREKP

SEQ ID NO: 46 - protein, 90 aa, Coronavirus
ALKFDGKVPVEFQLEVNQSRDNSRSRSQSRSQSRNRSQSRGRQQSNNKKDDSVEQAVLAALKKLGVDTEKQQQRSRSKSKERSNSKTRDT

SEQ ID NO: 47 - protein, 86 aa, Coronavirus
PLRFDGKIPPQFQLEVNRPRNNSRSGSQSRSVSRNRSQSRGRHHSNNQNNNVEDTIVAVLEKLGVTDKQRSRSKPRERSDSKPRDT

SEQ ID NO: 48 - protein, 82 aa, Coronavirus
PLRFAEGGPDDNFRWDFIPVGGRGRSGRSSVATSRESSRPGSREGSRGRRQMGPNEDLIARAARIIEEQQKRGGRITKAKAN

SEQ ID NO: 49 - protein, 80 aa, Coronavirus
PLRFSDGGPDGNFRWDFIPLSRGRSGRSTAVSSAASSRAPSREGSRGRRSGSVDDLIARAAKIIQDQQKKGSRITKAKAD

SEQ ID NO: 50 - protein, 88 aa, Coronavirus
PTRFAPGTVLPQGFYVEGSGRSAPASRSGSRSQSRGPNNRARSSSNQRQPASTVKPDMAEEIAALVLAKLGKDAGQPKQVTKQSAKEV

SEQ ID NO: 51 - protein, 88 aa, Coronavirus
PTRFAPGTVLPQGFYVEGSGRSAPASRSGSRSQSRGPSNRARSSSNQRQPASTVKPDMAEEIAALVLAKLGKDAGQPKQVTKQSAKEV

SEQ ID NO: 52 - protein, 91 aa, Coronavirus
PTRFAPGTVLPKGYYVEGSGRSAPSSRSSSRASSRASSVGGSRSRNNSASRNNTSEVTPDMADQIASLVLAKLNKESGKPVQVTKQTAKEI

SEQ ID NO: 53 - protein, 90 aa, Coronavirus
PTRFPPGTVLPQGYYIEGSGRSAPNSRSTSRTSSRASSAGSRSRANSGNRTPTSGVTPDMADQIASLVLAKLGKDATKPQQVTKHTAKEV

SEQ ID NO: 54 - protein, 90 aa, Coronavirus
PTRFPPGTVLPQGYYIEGSGRSAPNSRSTSRASSRASSAGSRSRANSGNRTPTSGVTPDMADQIVSLVLAKLGKDATKPQQVTKQTAKEI

SEQ ID NO: 55 - protein, 93 aa, Coronavirus
VIQLPQGTTLPKGFYAEGSRGGSQASSRSNSRSRGNSRNSTPSSSRGSSPARMAAGGDTALALLLLDRLNQLESKVSGKTPQQSQVVTKKTAA

SEQ ID NO: 56 - protein, 95 aa, Coronavirus
VLQLPQGTTLPKGFYAEGSRGGSQASSRSSSRSRGNSRNSTPGSSRGNSPARMASGGGETALALLLLDRLNQLESKVSGKGQQQQGQTVTKKSAA

SEQ ID NO: 57 - protein, 95 aa, Coronavirus
VLQLPQGTTLPKGFYAEGSRGGSQASSRSSSRSRNSSRNSTPGSSRGTSPARMAGNGGDAALALLLLDRLNQLESKMSGKGQQQQGQTVTKKSAA

SEQ ID NO: 58 - protein, 95 aa, Coronavirus
VLQLPQGTTLPKGFYAEGSRGGSQASSRSSSRSRNSSRNTTPGSSRGTSPARMAGNGGDAALALLLLDRLNQLESKMSGKGQQQQGQTVTKKSAA

SEQ ID NO: 59 - protein, 95 aa, Coronavirus
VTQFAPGTQLPKNCHIEGTGGNSQSSSRASSTSRNSTRSNSRGSQPGSRSHSPGNAVGPSDTSALLYLELVKRLDALEAGKTKSAPKVITKKDVS

SEQ ID NO: 60 - protein, 98 aa, Coronavirus
VTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAA

SEQ ID NO: 61 - protein, 100 aa, Coronavirus
VTQFAPGTKLPKNFHIEGTGGNSQSSSRASSASRGSSRSSSRGARSGNSSRSASPGPAGIGAVGGDASSILYLSLLKRLEDLEAGKSKSAPKVVTKKDAA